(19) United States
(12) Patent Application Publication
(10) Pub. No.: US 2021/0388415 A1
(43) Pub. Date: Dec. 16, 2021

(54) METHODS AND KITS FOR DEPLETION AND ENRICHMENT OF NUCLEIC ACID SEQUENCES

(71) Applicant: University of Washington, Seattle, WA (US)

(72) Inventors: Alexander B. Rosenberg, Seattle, WA (US); Charles Roco, Seattle, WA (US); Georg Seelig, Seattle, WA (US)

(73) Assignee: University of Washington, Seattle, WA (US)

(21) Appl. No.: 17/287,099

(22) PCT Filed: Oct. 24, 2019
(86) PCT No.: PCT/US2019/057939

§ 371 Date: Apr. 20, 2021

Related U.S. Application Data
(60) Provisional application No. 62/750,169, filed on Oct. 24, 2018.

(51) Int. Cl. C12Q 1/6806; C12N 9/22

(52) U.S. Cl. CPC C12Q 1/6806; C12N 9/22

(57) ABSTRACT

Kits and methods for enriching target nucleic acid sequences, such as nucleic acid molecules including the target nucleic acid sequence, and kits and methods for depleting target nucleic acid sequences, such as nucleic acid molecules including the target nucleic acid sequences. In an embodiment, the methods for enriching target nucleic acid sequences include selectively degrading single-stranded sample nucleic acid molecules, such as those that do not include the target nucleic acid sequences. In an embodiment, the methods for depleting target nucleic acid sequences include selectively degrading double-stranded sample nucleic acid molecules, such as those including the target nucleic acid sequence.

### CROSS-REFERENCE TO RELATED APPLICATION

This application claims the benefit of U.S. Provisional Application No. 62/750,169, filed Oct. 24, 2018, which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 70380_Seq_final_2019-10-24.txt. The text file is 14 KB; was created on Oct. 24, 2019; and is being submitted via EFS-Web with the filing of the specification.

### BACKGROUND

Complex biological samples, such as tissues, cells, cell lysates, serum, and the like, present challenges to determining sequences and concentrations of nucleic acid molecules bearing particular target sequences. Likewise, determining sequences and concentrations of nucleic acids from a set of barcoded molecules, such as single-cell RNA sequencing libraries present similar challenges.

Conventionally, sequencing methods, such as Sanger sequencing or Next-Generation Sequencing (NGS) methods are used to sequence nucleic acids in such complex samples and sequence libraries, where large numbers of excess sequences are generated in addition to those based upon a target sequence of interest. Additionally, where NGS methods are used, relatively high numbers of sequencing reads are used to achieve a desired sequencing depth.

Selectively enriching for target nucleic acid sequences or depleting non-target nucleic acid sequences in complex samples would simplify interpreting sequence data and reduce a number of reads needed to achieve a particular sequencing depth.

Accordingly, there is presently a need in the art to selectively remove some or all nucleic acid molecules that are not of interest or selectively increase a proportion of nucleic acid molecules that are of interest in complex mixtures, such as in preparation for sequencing. The present disclosure seeks to fulfill these needs and provides further related advantages.

### SUMMARY

Toward that end, in certain aspects, the present disclosure provides methods and kits for enriching target nucleic acid molecules. Correspondingly, in other aspects, the present disclosure provides methods and kits for depleting nucleic acid molecules that are not of interest.

In one aspect the present disclosure provides a method for enriching a target nucleic acid sequence. In an embodiment, the method comprises introducing to a sample solution, comprising a plurality of sample nucleic acid molecules each comprising a universal adaptor nucleic acid sequence, a capture primer nucleic acid molecule complementary to or partially complementary to a target nucleic acid sequence of one or more sample nucleic acid molecules of the plurality of sample nucleic acid molecules; enzymatically extending the capture primer nucleic acid molecule annealed to the target nucleic acid sequence of the one or more sample nucleic acid molecules; and enzymatically degrading single-stranded sample nucleic acid molecules, to provide an enriched sample solution having a higher proportion of sample nucleic acid molecules comprising the target nucleic acid sequence than the sample solution.

In another aspect, the present disclosure provides a method for depleting a target nucleic acid sequence. In an embodiment, the method comprises introducing to a sample solution, comprising a plurality of sample nucleic acid molecules each comprising a universal adaptor nucleic acid sequence comprising ribonucleotides, a capture primer nucleic acid molecule complementary or partially complementary to a target nucleic acid sequence of one or more sample nucleic acid molecules of the plurality of sample nucleic acid molecules; enzymatically extending the capture primer nucleic acid molecule annealed to the target nucleic acid sequence of the one or more sample nucleic acid molecules; and enzymatically cleaving double-stranded ribonucleic acid molecules of the sample nucleic acid molecules, to provide a depleted sample solution having a lower proportion of sample nucleic acid molecules comprising the target nucleic acid sequence than the sample solution.

In an aspect, the present disclosure provides a kit for enriching a target nucleic acid sequence. In an embodiment, the kit comprises a capture primer nucleic acid molecule complementary to or partially complementary to a target sequence; and a degradation enzyme configured to degrade a single-stranded nucleic acid molecule.

In another aspect, the present disclosure provides a kit for depleting a target nucleic acid sequence. In an embodiment the kit comprises a capture primer nucleic acid molecule complementary to or partially complementary to a target sequence; and a degradation enzyme configured to degrade a double-stranded nucleic acid molecule.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A schematically illustrates a sample solution including sample nucleic acid molecules to enrich and nucleic acid molecules to deplete, an accordance with an embodiment of the disclosure.

FIG. 1B schematically illustrates the sample solution of FIG. 1A further including capture primer nucleic acid molecules, in accordance with an embodiment of the disclosure.

FIG. 1C schematically illustrates the sample solution of FIG. 1B after melting the sample nucleic acid molecules to enrich and to deplete, in accordance with an embodiment of the disclosure.

FIG. 1D schematically illustrates the sample solution of FIG. 1C after annealing a capture primer nucleic acid molecule to a target sequence of a nucleic acid molecule to be enriched, in accordance with an embodiment of the disclosure.

FIG. 1E schematically illustrates the sample solution of FIG. 1D after enzymatically extending the capture primer nucleic acid molecule annealed to the target sequence, in accordance with an embodiment of the disclosure.

FIG. 1F schematically illustrates the sample solution of FIG. 1E after enzymatically degrading single-stranded sample nucleic acid molecules in the sample solution, in accordance with an embodiment of the disclosure.

FIG. 1G schematically illustrates melting the nucleic acid molecules of the sample solution of FIG. 1F, in accordance with an embodiment of the disclosure.

FIG. 1H schematically illustrates the sample solution of FIG. 1G after removing the capture primer nucleic acid molecules, in accordance with an embodiment of the disclosure.

FIG. 1I schematically illustrates the sample solution of FIG. 1H further including polymerase chain reaction (PCR) primers complementary to a universal adaptor sequence of the sample nucleic acid molecules in the sample solution, in accordance with an embodiment of the disclosure.

FIG. 1J schematically illustrates the sample solution of FIG. 1I after PCR amplification of certain sample nucleic acid molecules of the sample solution, in accordance with an embodiment of the disclosure.

FIG. 2A schematically illustrates a sample solution including sample nucleic acid molecules to enrich and nucleic acid molecules to deplete, in accordance with an embodiment of the disclosure.

FIG. 2B schematically illustrates the sample solution of FIG. 2A further including capture primer nucleic acid molecules, in accordance with an embodiment of the disclosure.

FIG. 2C schematically illustrates the sample solution of FIG. 2B after melting the sample nucleic acid molecules to enrich and to deplete, in accordance with an embodiment of the disclosure.

FIG. 2D schematically illustrates the sample solution of FIG. 2C after annealing a capture primer nucleic acid molecule to a target sequence of a nucleic acid molecule to be depleted, in accordance with an embodiment of the disclosure.

FIG. 2E schematically illustrates the sample solution of FIG. 2D after enzymatically extending the capture primer nucleic acid molecule annealed to the target sequence, in accordance with an embodiment of the disclosure.

FIG. 2F schematically illustrates the sample solution of FIG. 2E after enzymatically degrading double-stranded sample nucleic acid molecules in the sample solution, in accordance with an embodiment of the disclosure.

FIG. 2G schematically illustrates melting the sample nucleic acid molecules of the sample solution of FIG. 2F, in accordance with an embodiment of the disclosure.

FIG. 2H schematically illustrates the sample solution of FIG. 2G after removing the capture primer nucleic acid molecules, in accordance with an embodiment of the disclosure.

FIG. 2I schematically illustrates the sample solution of FIG. 2H further including PCR primers complementary to a universal adaptor sequence of the sample nucleic acid molecules in the sample solution, in accordance with an embodiment of the disclosure.

FIG. 2J schematically illustrates the sample solution of FIG. 2I after PCR amplification of the sample nucleic acid molecules of the sample solution, in accordance with an embodiment of the disclosure.

FIG. 3A schematically illustrates a sample solution including sample nucleic acid molecules to enrich and nucleic acid molecules to deplete, in accordance with an embodiment of the disclosure.

FIG. 3B schematically illustrates the sample solution of FIG. 3A further including capture primer nucleic acid molecules including blocked capture primer nucleic acid molecules, in accordance with an embodiment of the disclosure.

FIG. 3C schematically illustrates the sample solution of FIG. 3B after melting the sample nucleic acid molecules to enrich and to deplete, in accordance with an embodiment of the disclosure.

FIG. 3D schematically illustrates the sample solution of FIG. 3C after annealing a capture primer nucleic acid molecule to a target sequence of a nucleic acid molecule to be depleted, in accordance with an embodiment of the disclosure.

FIG. 3E schematically illustrates the sample solution of FIG. 3D after enzymatically extending the capture primer nucleic acid molecule annealed to the target sequence, in accordance with an embodiment of the disclosure.

FIG. 3F schematically illustrates the sample solution of FIG. 3E after enzymatically degrading double-stranded sample nucleic acid molecules in the sample solution, in accordance with an embodiment of the disclosure.

FIG. 3G schematically illustrates melting the sample nucleic acid molecules of the sample solution of FIG. 2F, in accordance with an embodiment of the disclosure.

FIG. 3H schematically illustrates the sample solution of FIG. 3G after removing the capture primer nucleic acid molecules, in accordance with an embodiment of the disclosure.

FIG. 3I schematically illustrates the sample solution of FIG. 3H further including PCR primers complementary to a universal adaptor sequence of the sample nucleic acid molecules in the sample solution, in accordance with an embodiment of the disclosure.

FIG. 3J schematically illustrates the sample solution of FIG. 3I after PCR amplification of the sample nucleic acid molecules of the sample solution, in accordance with an embodiment of the disclosure.

FIG. 4 schematically illustrates capture primer nucleic acid molecules, in accordance with an embodiment of the disclosure, bound to Hygro.

FIG. 5 schematically illustrates capture primer nucleic acid molecules, in accordance with an embodiment of the disclosure, bound to AmpR FIG. 6 schematically illustrates capture primer nucleic acid molecules including a universal adaptor sequence including a polyT sequence, in accordance with an embodiment of the disclosure, bound to AmpR.

FIG. 7 schematically illustrates capture primer nucleic acid molecules molecules including a universal adaptor sequence including a polyT sequence, in accordance with an embodiment of the disclosure, bound to Hygro.

FIG. 8 is an image of an electrophoresis gel showing results of an electrophoresis experiment showing enrichment of sample nucleic acid molecules including a target sequence, in accordance with an embodiment of the disclosure.

### DETAILED DESCRIPTION

The present disclosure provides kits and methods for enriching target nucleic acid sequences, such as nucleic acid molecules including the target nucleic acid sequence, and kits and methods for depleting target nucleic acid sequences, such as nucleic acid molecules including the target nucleic acid sequences.

As used herein, the terms “nucleic acid” and “polynucleotides” refer to biopolymers that are made from monomer units referred to as “nucleotides.” Typically, each nucleotide is composed of a 5-carbon sugar, a phosphate group, and a nitrogenous base (also referred to as “nucleobase”). The structure of the sugar component typically defines to the type of nucleic acid polymer. The nucleotide monomers link up to form a linear sequence of the nucleic acid polymer. Nucleic acids encompassed by the present disclosure can include deoxyribonucleic acid (DNA), ribonucleic acid (RNA), cDNA or a synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains, or any combination thereof. Nucleic acid molecules can be single stranded or double stranded (with complementary single-stranded polynucleotide chains hybridizing by base pairing of the individual nucleobases). Typically cDNA, RNA, GNA, TNA or LNA are single stranded. DNA can be either double stranded (dsDNA) or single stranded (ssDNA).

Nucleotide subunits of nucleic acids can be naturally occurring, artificial, or modified. As indicated above, nucleotide typically contains a nucleobase, a sugar, and at least one phosphate group. The nucleobase is typically heterocyclic. Suitable nucleobases include the canonical purines and pyrimidines, and more specifically adenine (A), guanine (G), thymine (T) (or typically in RNA, uracil (U) instead of thymine (T)), and cytosine (C). The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate. These are generally referred to herein as nucleotides or nucleotide residues to indicate the subunit. Without specific identification, the term nucleotides, nucleotide residues, and the like, is not intended to imply any specific structure or identity. As indicated above, the nucleic acids of the present disclosure can also include synthetic variants of DNA or RNA. “Synthetic variants” encompasses nucleic acids incorporating known analogs of natural nucleotides/nucleobases that can hybridize to nucleic acids in a manner similar to naturally occurring nucleotides. Exemplary synthetic variants include peptide nucleic acids (PNAs), phosphorothioate DNA, locked nucleic acids, and the like. Modified or synthetic nucleobases and analogs can include, but are not limited to, 5-Br-UTP, 5-Br-dUTP, 5-F-UTP, 5-F-dUTP, 5-propynyl dCTP, 5-propynyl-dUTP, diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N 6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Persons of ordinary skill in the art can readily determine what base pairings for each modified nucleobase are deemed a base-pair match versus a base-pair mismatch.

### Methods

In an aspect, the present disclosure provides methods for enriching and/or depleting target nucleic acid sequences, such as target nucleic acid sequences present on sample nucleic acids in a in complex sample solutions comprising sample nucleic acid molecules that do not include the target nucleic acid sequence.

Enrichment Methods

In an embodiment, the present disclosure provides method for enriching a target nucleic acid sequence. In an embodiment, the method for enriching a target nucleic acid sequence comprises (a) introducing to a sample solution, comprising a plurality of sample nucleic acid molecules each comprising a universal adaptor nucleic acid sequence, a capture primer nucleic acid molecule complementary to or partially complementary to a target nucleic acid sequence of one or more sample nucleic acid molecules of the plurality of sample nucleic acid molecules; (b) enzymatically extending the capture primer nucleic acid molecule annealed to the target nucleic acid sequence of the one or more sample nucleic acid molecules; and (c) enzymatically degrading single-stranded sample nucleic acid molecules, to provide an enriched sample solution having a higher proportion of sample nucleic acid molecules comprising the target nucleic acid sequence than the sample solution.

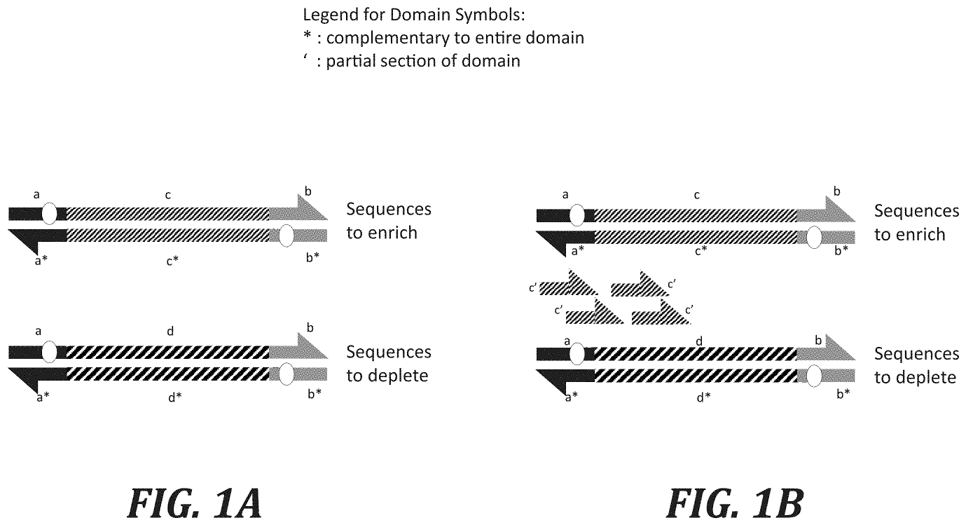

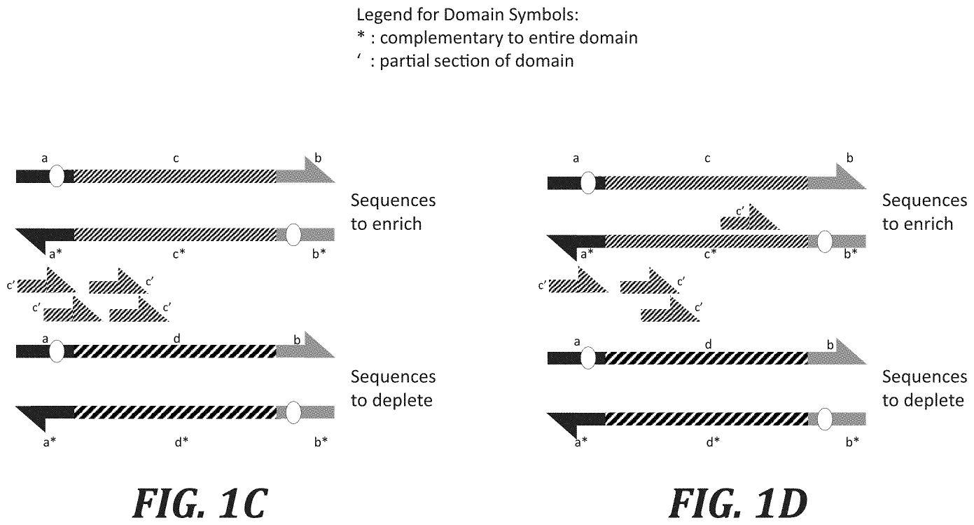

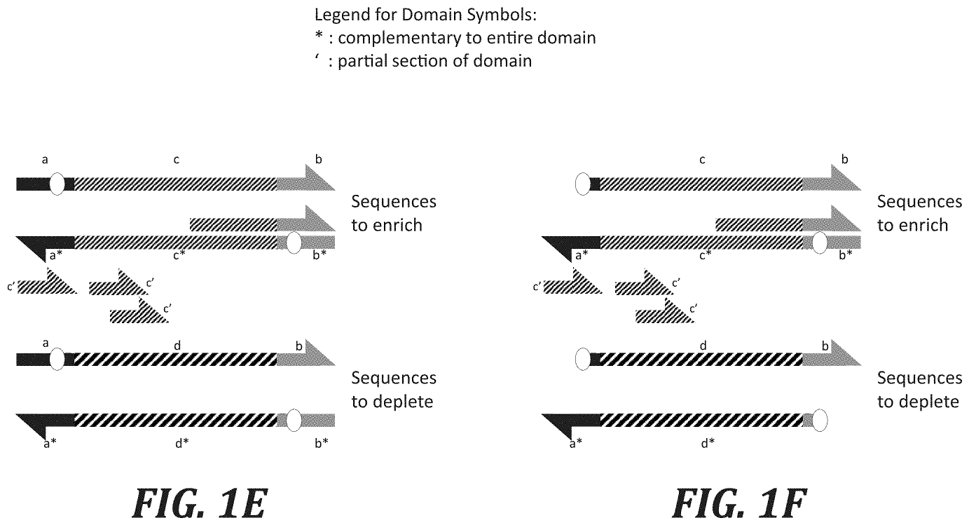

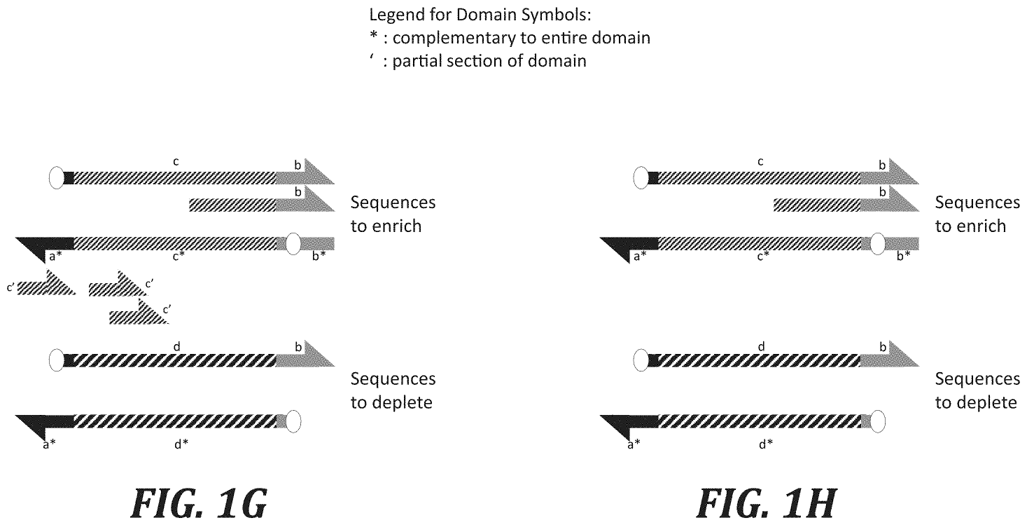

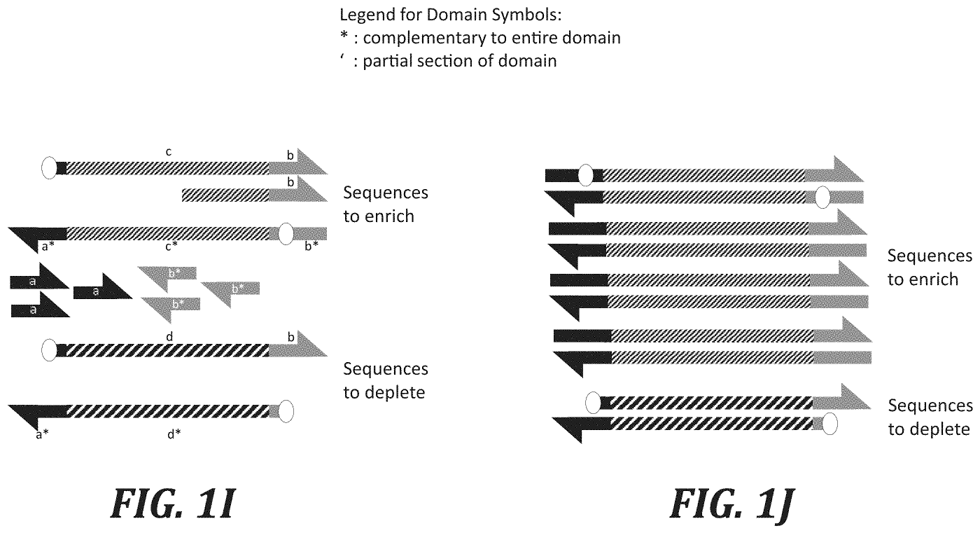

A method for enriching target nucleic acid sequences in accordance with an embodiment of the disclosure will now be described. In that regard, attention is directed to FIGS. 1A-1J, which schematically illustrates a method of enriching a target nucleic acid sequence, in accordance with an embodiment of the disclosure.

FIG. 1A schematically illustrates a sample solution including nucleic acid molecules to enrich and nucleic acid molecules to deplete. As shown, the sample solution includes a starting pool of nucleic acid molecules including a double-stranded nucleic acid molecule for enrichment and a double stranded nucleic acid molecule for depletion. While the sample nucleic acid molecules are shown to be double stranded, in an embodiment, the sample nucleic acid molecules include single-stranded sample nucleic acid molecules or a combination of single-stranded and double-stranded sample nucleic acid molecules. The double-stranded nucleic acid molecule for enrichment is shown to include universal adaptor nucleic acid sequences a, a*, b, and b*, and target nucleic acid sequences c and c*. The double-stranded nucleic acid molecule for depletion is shown to include universal adaptor nucleic acid sequences a, a*, b, and b*, and nucleic acid sequences d and d* different from the target nucleic acid sequences c and c*. The universal adaptor nucleic acid sequences a, a*, b, and b*, on both the sample nucleic acid molecules for enrichment and for depletion, are shown to include a common feature, illustrated schematically here as an oval. As discussed further herein with respect to FIG. 1F, such a common feature is suitable for enzymatic degradation under certain conditions, such as where the universal adaptor nucleic acid sequence is single stranded.

The methods of the present disclosure are suitable to enrich a number of sample solutions comprising nucleic acid molecules. In an embodiment, the sample solution is selected from the group consisting of a WGS library, a WES library, ATAC-seq library, ChIP-seq library, WTS library, Bisulfite-seq library, RNA-seq library, single-cell RNA-seq library, DNA data storage library, or any other library with universal adapters on both ends. The mixture of DNA molecules can be previously amplified or unamplified, generated enzymatically or chemically synthesized. The universal adapters (domain a and domain b*) can include DNA and/or RNA nucleotides. As discussed further herein, at least one of the ribonucleotides may be a guanine. In an embodiment, the universal adaptor nucleic acid sequences present on all or substantially all nucleic acid molecules in the library.

In an embodiment, the sample solution includes double- or single-stranded sample nucleic acid molecules, such as from a WGS library, a WES library, ATAC-seq library, CHIP-seq library, WTS library, Bisulfite-seq library, RNA-seq library, and the like, containing 3′ modifications configured to prevent or limit self-annealing and extension. In an embodiment, such 3′ modifications include dideoxynucleotides (ddNTPs), inverted 3′dT, or nucleotide sequences that reduce binding energy (e.g. adenine, thymine, or uracil). In one embodiment, the starting sample solution includes double-stranded sample nucleic acid molecules, such as a WGS library, a WES library, ATAC-seq library, CHIP-seq library, WTS library, Bisulfite-seq library, RNA-seq library, and the like, generated by using PCR primers that contain polyT or polyA overhangs on the 5′ end.

In an embodiment, the universal adaptor nucleic acid sequences are added through PCR, transposition, reverse transcription, ligation, chemical synthesis, or other known methods to add adapters to DNA sequences, such as discussed further herein with respect to the kits of the present disclosure.

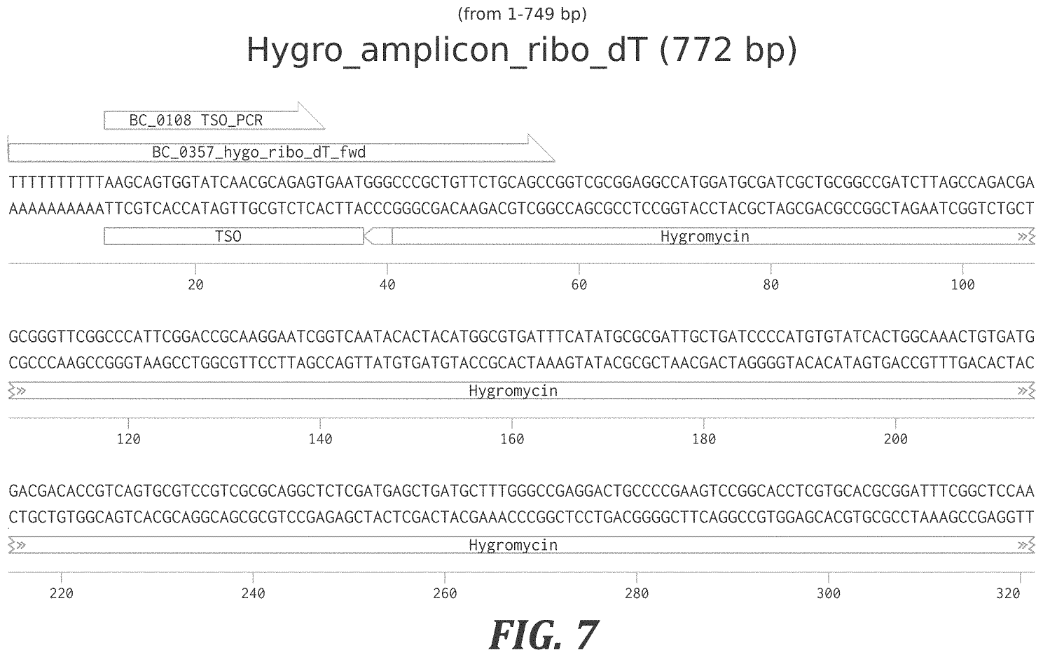

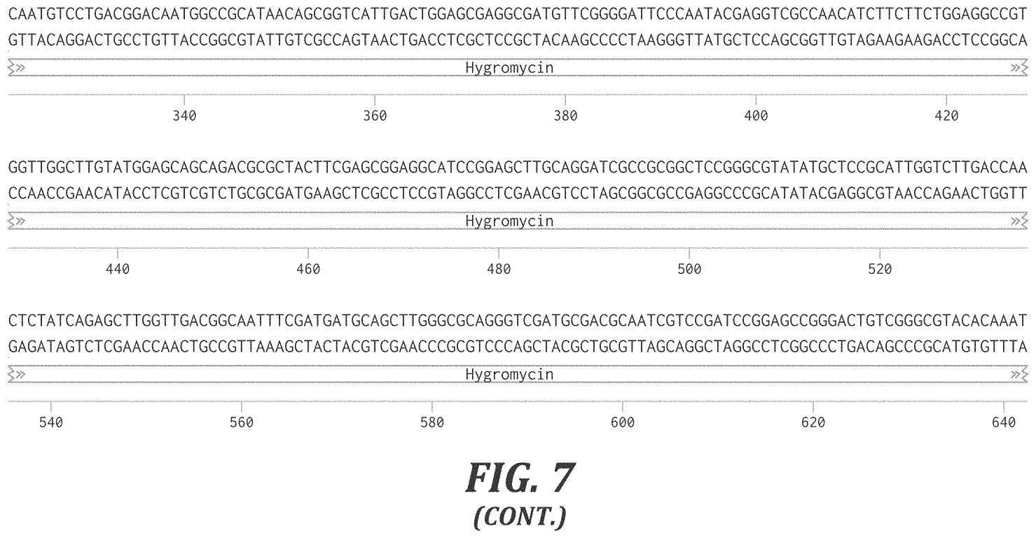

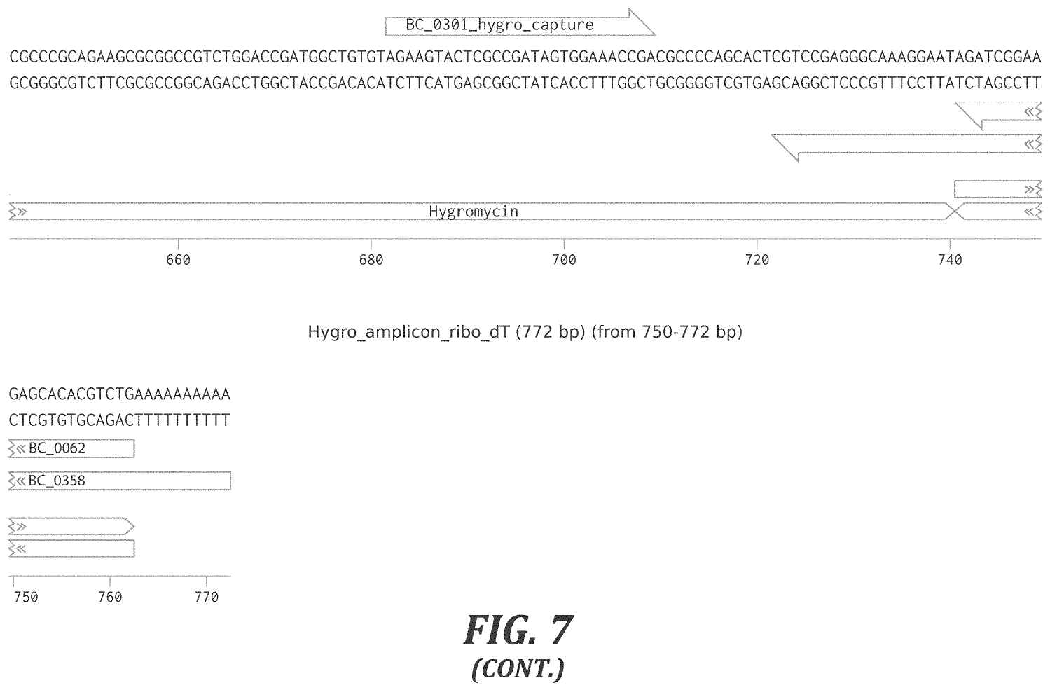

In an embodiment, the universal adaptor nucleic acid sequence includes a nucleic acid sequence adjacent to a 3′ end or a 5′ end that is configured not to bind to itself, such as in a hairpin configuration, thus avoiding self-priming. In an embodiment, the universal adaptor nucleic acid molecule includes a polyT sequence, a polyA sequence, or a combination thereof. See for example, FIGS. 6 and 7.

In an embodiment, nucleotides in the sample solution include ribonucleotides or deoxynucleotides. In an embodiment, such nucleotides include nucleotides selected from the group consisting of locked nucleic acids, peptide nucleic acids, 2′-O-methyl RNA, 2′-O:-methoxy ethyl RNA, phosphorothioate modified nucleic acids, and the like. Accordingly, in an embodiment, the degradation enzyme discussed further herein, such as, RNase T1, is replaced by a degradation enzyme capable of selectively cleaving the modified ribonucleotide or deoxynucleotide in a single-stranded conformation.

As above, in an embodiment, the method includes introducing to the sample solution one or more capture primer nucleic acid molecule(s) complementary to or partially complementary to a target nucleic acid sequence of one or more sample nucleic acid molecules of the plurality of sample nucleic acid molecules. FIG. 1B schematically illustrates the sample solution of FIG. 1A further including capture primer nucleic acid molecules c′, in accordance with an embodiment of the disclosure.

As above, the capture primer nucleic acid molecule is complementary to or partially complementary to the target nucleic acid sequence. In an embodiment, the capture primer nucleic acid molecule is partially complementary to the target nucleic acid sequence. In an embodiment, the capture primer nucleic acid molecule comprises a number of bases that are not complementary to the target nucleic acid sequence, such as in a range of 1 to 5. In an embodiment, the capture primer nucleic acid molecule is greater than or equal to 90% complementary to the target nucleic acid sequence. Such partially complementary capture primer nucleic acid molecules are, nevertheless, configured to bind with target nucleic acid sequences, such as depending upon the annealing temperatures and/or other reaction conditions described herein.

In an embodiment, the method includes maintaining a temperature of the sample solution at or above a melting temperature of the plurality of sample nucleic acid molecules. FIG. 1C schematically illustrates the sample solution of FIG. 1B after melting the nucleic acid molecules to enrich and to deplete, in accordance with an embodiment of the disclosure. In an embodiment, the melting temperature is greater than or equal to 95° C. At the melting temperature of the plurality of sample nucleic acid molecules the temperature of the sample solution is sufficient to completely or partially break Watson-Crick bonding between sample nucleic acid molecules, thereby increasing the number of single-stranded or partially single-stranded sample nucleic acid molecules in the sample solution. As shown, such melting exposes target nucleic acid sequences c and c*, as well as nucleic acid sequences d and d*, to bonding with other nucleic acid sequences, such as the capture primer nucleic acid molecules, c′.

In an embodiment, the method includes maintaining the sample solution at about or below an annealing temperature of the capture primer nucleic acid molecule suitable to anneal the capture primer nucleic acid molecule to the target nucleic acid sequence. Such an annealing temperature is generally suitable to anneal at least a portion of the capture primer nucleic acid molecules to the target nucleic acid sequence. In an embodiment, the annealing temperature is in a range of about 50° C. to about 72° C. FIG. 1D schematically illustrates the sample solution of FIG. 1C after annealing a capture primer nucleic acid molecule c′ to a target sequence c* of a nucleic acid molecule to be enriched, in accordance with an embodiment of the disclosure. In the illustrated embodiment, one of the capture primer nucleic acid molecules c′ is bound to the target nucleic acid sequence c* of a sample nucleic acid molecule to be enriched.

In an embodiment, the capture primer nucleic acid molecule is configured to be primarily single stranded at the annealing temperature. In this regard, the capture primer nucleic acid molecule is single stranded a majority of the time at the annealing temperature, and is, therefore, configured to bind to the target nucleic acid sequence a majority of the time. In an embodiment, the capture primer nucleic acid molecule is configured to be primarily at least partially double stranded at the annealing temperature. In this regard, the capture primer nucleic acid molecule is in a configuration suitable for binding to a target nucleic acid sequence less than a majority of the time at the annealing temperature. Thus, binding of such a double-stranded capture primer nucleic acid molecule to a target nucleic acid sequence is generally more selective than for single-stranded capture primer nucleic acid molecules.

In an embodiment, the capture primer nucleic acid molecule further comprises a second capture primer nucleic acid molecule complementary to or partially complementary to a first capture primer nucleic acid molecule. Such double-stranded capture primer nucleic acid molecules are generally double stranded at the annealing temperature and are, thus, less often configured to bind to a target nucleic acid sequence. In this regard, such double-stranded capture primer nucleic acid molecules are configured to bind more selectively to target nucleic acid sequences.

In an embodiment, the capture primer nucleic acid molecule is complementary to or partially complementary to a second target nucleic acid sequence of one or more second sample nucleic acid molecules of the plurality of sample nucleic acid molecules, wherein the second target nucleic acid sequence is different than the target nucleic acid sequence. In this regard, by maintaining the sample solution at or at about an annealing temperature of the capture primer nucleic acid molecule, the capture primer nucleic acid molecules may bind to various target nucleic acid sequences. As discussed further herein with respect to FIGS. 1E and 1F, sample nucleic acid molecules comprising various target sequences complementary to or partially complementary to the capture primer nucleic acid molecules may be enzymatically extended and protected from degradation.

In an embodiment, the capture primer nucleic acid molecule comprises a phosphorothioate linkage. In an embodiment, the phosphorothioate linkage is disposed between a base at a 3′ end of the capture primer nucleic acid molecule and a base immediately adjacent to the base at the 3′ end. Such phosphorothioate linkages are configured to resist 3′ exonuclease activity, such as those present in proof reading polymerases.

As above, the sample nucleic acid molecules include a universal adaptor nucleic acid sequence. In an embodiment, the universal adaptor nucleic acid sequence of the plurality of sample nucleic acid molecules comprises an adaptor tag nucleic acid sequence. In an embodiment, the adaptor tag nucleic acid sequence defines a unique nucleic acid sequence. Such a unique sequence can be used to determine an origin of the sample nucleic acid molecules, such as a cell, tissue, or suspension of origin, where such unique nucleic acid sequences have different sequences from another adaptor tag nucleic acid sequence used to tag sample nucleic acid molecules in other samples, such as in other cells, tissues, or suspensions of cells.

Such adaptor tag nucleic acid sequences are suitable for counting a number of nucleic acid molecules in a sample, such as through sequencing the sample solution. In an embodiment, each adaptor tag nucleic acid molecule includes a number of degenerate bases suitable for counting amplified sample nucleic acid molecules after a nucleic acid amplification reaction.

In an embodiment, an annealing temperature of the capture primer nucleic acid molecule and the second target nucleic acid sequence is relatively close to the annealing temperature of the capture primer nucleic acid molecule and the target nucleic acid sequence, such that by maintaining the sample solution at the annealing temperature of the capture primer nucleic acid molecule and the target nucleic acid sequence, at least some of the capture primer nucleic acid molecules bind to the second target nucleic acid sequence. Accordingly, in an embodiment, the capture primer nucleic acid molecule and the second target nucleic acid sequence have a second annealing temperature in a range of about 1° C. to about 5° C. of the annealing temperature.

In an embodiment, the sample solution is maintained at temperatures that are near, but not necessarily precisely at, the annealing temperature. In this regard, the binding specificity of the capture primer nucleic acid molecules is varied, allowing the capture primer nucleic acid molecules to bind, for example, to a number of target nucleic acid sequences having relatively similar sequences, and thus enriching a number of different sample nucleic acid molecules. Accordingly, in an embodiment, maintaining the sample solution at about or below an annealing temperature of the capture primer nucleic acid molecule comprises maintaining the sample solution at a temperature within a range of about 1° C. to about 5° C. of the annealing temperature of the capture primer nucleic acid molecule.

As above, in an embodiment, the methods of the present disclosure include enzymatically extending the capture primer nucleic acid molecule annealed to the target nucleic acid sequence of the one or more sample nucleic acid molecules. In an embodiment, enzymatically extending the capture primer nucleic acid molecule comprises introducing to the sample solution an extension enzyme configured to extend the capture primer nucleic acid molecule annealed to the target nucleic acid sequence. FIG. 1E schematically illustrates the sample solution of FIG. 1D after enzymatically extending the capture primer nucleic acid molecule c′ annealed to the target sequence c*, in accordance with an embodiment of the disclosure. As shown, the nucleic acid sequence annealed to the target nucleic acid sequence c* is shown extended to also bind with the universal adaptor nucleic acid sequence b*. As discussed further herein, by binding to the universal adaptor nucleic acid sequence, the extended capture primer nucleic acid molecule inhibits enzymatic degradation of the double-stranded sample nucleic acid molecule.

The extension enzyme can include any enzyme configured to enzymatically extend the capture primer nucleic acid molecule annealed to another nucleic acid molecule. In an embodiment, the extension enzyme is selected from the group consisting of a polymerase, a reverse transcriptase, and combinations thereof.

In an embodiment, enzymatically extending the capture primer nucleic acid molecule comprises maintaining the sample solution at about an extension temperature of the extension enzyme suitable for enzymatic extension by the extension enzyme of the capture primer nucleic acid molecule annealed to the target nucleic acid sequence. Such an extension temperature may be the same as or different from the annealing temperature. In an embodiment, the extension temperature is in a range of about 68° C. to about 72° C.

The methods of the present disclosure include enzymatically degrading certain nucleic acid molecules of the sample solution to provide an enriched sample solution having a higher proportion of sample nucleic acid molecules comprising the target nucleic acid sequence(s) than the sample solution. In an embodiment, such enzymatic degradation includes enzymatically degrading single-stranded sample nucleic acid molecules. As discussed above with respect to FIGS. 1D and 1E, sample nucleic acid molecules including nucleic acid sequences complementary to or partially complementary to the capture primer nucleic acid molecules may be generally double-stranded. In this regard, by enzymatically degrading single-stranded nucleic acid molecules, such as in conjunction with other steps such as amplifying the intact sample nucleic acid molecules, the sample solution is enriched for such nucleic acid molecules including target nucleic acid sequences.

In an embodiment, enzymatically degrading single-stranded sample nucleic acid molecules comprises introducing to the sample solution a degradation enzyme configured to degrade a single-stranded nucleic acid molecule comprising the universal adaptor nucleic acid sequence. In an embodiment, the degradation enzyme is introduced to the sample solution after enzymatically extending the capture primer nucleic acid molecule. In an embodiment, wherein the degradation enzyme is introduced to the sample solution before enzymatically extending the capture primer nucleic acid molecule. In such an embodiment, the degradation enzyme may not be active at, for example, at the extension temperature, and, therefore, does not or does not substantially degrade single-stranded nucleic acid molecules at the extension temperature. Rather, in an embodiment, the degradation enzyme is active a temperature lower than the extension temperature.

In an embodiment, enzymatically degrading single-stranded sample nucleic acid molecules comprises maintaining the temperature of the sample solution at a degradation temperature of the degradation enzyme. In an embodiment, the degradation temperature is below the annealing temperature. In an embodiment, the degradation temperature is below the extension temperature. In an embodiment, the degradation temperature is less than or equal to about 60° C.

In an embodiment, the degradation temperature is an active temperature of the degradation enzyme. Accordingly, by maintaining the sample solution at or at about the degradation, the degradation enzyme is active, such as active in degrading single-stranded nucleic acid molecules. In an embodiment, the degradation enzyme is inactive at a temperature chosen from the extension temperature, the melting temperature, the annealing temperature, and combinations thereof. In this regard, the degradation enzyme does not or does not substantially enzymatically degrade single-stranded nucleic acid molecules in the sample solution, such as before enzymatic extension of annealed capture primer nucleic acid molecules annealed to the target nucleic acid sequences.

In an embodiment, the degradation enzyme is active at the degradation temperature after being inactive at a temperature above the degradation temperature, such as the extension temperature. In this regard, in an embodiment, the degradation enzyme is configured to preferentially or selectively degrade sample nucleic acid molecules, such as single-stranded sample nucleic acid molecules, after having been inactive at a temperature above the degradation temperature. Without wishing to be bound by theory, it is believed that the degradation enzyme is inactive above the active temperature, such as when the degradation enzyme takes on an inactive conformation, and that the degradation further becomes active when the degradation enzyme assumes an active configuration when the temperature of the sample solution is maintained in an active range.

In an embodiment, enzymatically degrading the single-stranded sample nucleic acid molecules includes degrading a portion of the universal adaptor nucleic acid sequence disposed on the single-stranded sample nucleic acid molecules. FIG. 1F schematically illustrates the sample solution of FIG. 1E after enzymatically degrading the single-stranded sample nucleic acid molecules, in accordance with an embodiment of the disclosure. In the illustrated embodiment, the degradation enzyme is shown to have enzymatically degraded a portion of the single-stranded nucleic acid molecule including the universal adaptor sequence b*, formerly including the targeted portion of the universal adaptor nucleic acid sequence (illustrated here as an oval). This is in contrast to the double-stranded sample nucleic acid, which includes the target nucleic acid sequence c* and has been enzymatically extended by the extension enzyme. In this regard, the double-stranded sample nucleic acid is shown to have an intact universal adaptor nucleic acid sequence b*.

In an embodiment, the universal adaptor nucleic acid sequence is entirely single stranded. In this regard, the universal adaptor nucleic acid sequence is not base paired with other nucleic acid sequences, such as on separate nucleic acid molecules. In an embodiment, the universal adaptor nucleic acid sequence is only partially single stranded. In an embodiment, the universal adaptor nucleic acid sequence is single stranded at one or more nucleotides configured to be enzymatically degraded by the degradation enzyme when single stranded.

Enzymatic degradation of the single-stranded sample nucleic acid molecules can include a number of forms of degradation configured, for example, to make the degraded sample nucleic acid unsuitable for nucleic acid amplification reactions, such as those including the universal adaptor nucleic acid molecules. In an embodiment, enzymatically degrading the single-stranded sample nucleic acid molecules includes cleaving a backbone of the universal adaptor nucleic acid molecule of the single-stranded sample nucleic acid molecules. In an embodiment, enzymatically degrading the single-stranded sample nucleic acid molecules includes digesting a portion of the universal adaptor nucleic acid molecule of the single-stranded sample nucleic acid molecules.

As above, in an embodiment, the degradation enzyme is configured to enzymatically degrade single-stranded nucleic acid molecules, such as single-stranded sample nucleic acid molecules. In an embodiment, the degradation enzyme is a ribonuclease. In an embodiment, the degradation enzyme is an endonuclease. In an embodiment, the endonuclease is an endoribonuclease. In an embodiment, the endoribonuclease is selected from the group consisting of Rnase T1, Rnase A, and combinations thereof.

In an embodiment, the degradation enzyme is Rnase T1. In an embodiment, the degradation enzyme is according to SEQ ID NO. 14. In an embodiment, the degradation has a sequence homology to SEQ ID NO. 14 greater than 90%, greater than 95%, or greater than 99%. In an embodiment, the universal adaptor nucleic acid sequence comprises a riboguanine. In an embodiment, the universal adaptor nucleic acid sequence comprises a plurality of riboguanines. Rnase T1 selectively degrades single-stranded riboguanines, and, accordingly, where the universal adaptor nucleic acid sequence includes one or more riboguanines, the Rnase T1 degradation enzyme is configured to degrade the universal adaptor nucleic acid sequence, such as when the sample solution is maintained at an active temperature of Rnase T1.

In an embodiment, the degradation enzyme is Rnase A. In an embodiment, the degradation enzyme is according to SEQ ID NO. 15. In an embodiment, the degradation has a sequence homology to SEQ ID NO. 15 greater than 90%, greater than 95%, or greater than 99%. In an embodiment, the universal adaptor nucleic acid sequence comprises bases selected from the group consisting of a ribocytosine, a ribouracil, and combinations thereof. In an embodiment, the universal adaptor nucleic acid sequence comprises a plurality of ribocytosines, a plurality of ribouracils, and combinations thereof. Rnase A selectively degrades single-stranded ribocytosines and ribouracils (such as at salt concentrations above 300 mM), and accordingly, where the universal adaptor nucleic acid sequences includes one or more ribocytosines and/or ribouracils, the Rnase A degradation enzyme is configured to degrade the universal adaptor nucleic acid sequence, such as when the sample solution is maintained at an active temperature of Rnase A.

In an embodiment, the method of the present disclosure includes repeating enzymatically extending the capture primer nucleic acid molecule and enzymatically degrading single-stranded sample nucleic acid molecules. By repeating enzymatic extension and enzymatic degradation, the extension enzyme, capture primer nucleic acid molecules, and degradation enzyme can be used one or more additional times to selectively degrade sample nucleic acid molecules that do not include a target nucleic acid sequence. As above, in an embodiment, such degradation includes degrading the universal adaptor nucleic acid sequence, which can be later used in a nucleic acid amplification reaction. As discussed further herein with respect to FIGS. 1I and 1J, sequences that include intact universal adaptor nucleic acid sequences are preferentially enriched.

In an embodiment, the method further includes maintaining the temperature of the sample solution at or above a melting temperature of the plurality of sample nucleic acid molecules and the capture primer nucleic acid molecule, such as after enzymatically extending the capture primer nucleic acid molecule and enzymatically degrading single-stranded sample nucleic acid molecules. In this regard, the sample solution including sample nucleic acid molecules having enzymatically degraded or intact universal adaptor nucleic acid sequences are single stranded and, thus configured for further enzymatic extension and degradation. FIG. 1G schematically illustrates melting the nucleic acid molecules of the sample solution of FIG. 1F, in accordance with an embodiment of the disclosure.

In an embodiment, the method of the present disclosure includes purifying the plurality of sample nucleic acid molecules in the enriched sample solution. FIG. 1H schematically illustrates the sample solution of FIG. 1G after removing the capture primer nucleic acid molecules, in accordance with an embodiment of the disclosure. Such purification can include, for example, purification with SPRI beads and the like. In an embodiment, purifying the plurality of sample nucleic acid molecules in the enriched sample solution comprises removing reagents chosen from capture primer nucleic acid molecules, enzymes, and combinations thereof from the enriched sample solution. Such purification of the sample solution can simplify sequencing data based on the sample solution, such as by reducing the number of nucleic acid molecules present in the sample solution and, thereby, decreasing an amount of sequencing data based on the sample solution, particularly reducing an amount of sequencing data not related to target nucleic acid sequences.

In an embodiment, the method of the present disclosure includes amplifying sample nucleic acid molecules after enzymatic degradation of single-stranded nucleic acid molecules. Accordingly, in an embodiment the method includes introducing a plurality of amplification primer nucleic acid molecules to the enriched sample solution. In an embodiment, the amplification primer nucleic acid molecules of the plurality of amplification primer nucleic acid molecules are complementary to the universal adaptor nucleic acid sequence. FIG. 1I schematically illustrates the sample solution of FIG. 1H further including polymerase chain reaction (PCR) primers a* and b. As shown, the PCR primers are complementary to the universal adaptor sequences a* and b of the sample nucleic acid molecules in the sample solution, in accordance with an embodiment of the disclosure.

In an embodiment, the method includes performing a nucleic acid amplification reaction on the plurality of sample nucleic acid molecules in the enriched sample solution with the plurality of amplification primer nucleic acid molecules to provide an amplified enriched sample solution. FIG. 1J schematically illustrates the sample solution of FIG. 1I after PCR amplification of the sample nucleic acid molecules of the sample solution, in accordance with an embodiment of the disclosure. As shown, the sample solution includes a greater proportion of sample nucleic acid molecules including the target sequences c and c* than nucleic acid sequences d and d*.

As discussed above and shown in FIG. 1J, because at least some of the universal adaptor nucleic acid sequences of the sample nucleic acid molecules are degraded, these degraded sample nucleic acid molecules will not participate in the nucleic acid amplification reaction, and thus the amplified enriched sample solution will contain a lower proportion of such sample nucleic acid molecules. In this regard, in an embodiment, performing the nucleic acid amplification reaction on the plurality of sample nucleic acid molecules in the enriched sample solution does not or does not substantially amplify sample nucleic acid molecules that have been degraded by the degradation enzyme.

In an embodiment, the method includes performing one or more enzymatic reactions on the amplified enriched sample to solution to prepare the enriched sample solution for sequencing, such as a next-generation sample preparation. Accordingly, in an embodiment, the method of the present disclosure includes performing a reaction on the amplified enriched sample solution chosen from a nucleic acid fragmentation reaction, enzymatic end repair, A tailing, adaptor ligation, polymerase chain reaction, and combinations thereof.

In an embodiment, the method of the present disclosure includes sequencing nucleic acid molecules in the enriched sample solution. In an embodiment, sequencing nucleic acid molecules in the enriched sample solution comprises generating sample nucleic acid information based upon the plurality of sample nucleic acid molecules in the enriched sample solution. As above, in certain embodiment, the universal adaptor nucleic acid molecules include an adaptor tag nucleic acid molecule. In an embodiment, sequencing nucleic acid molecules in the enriched sample solution comprises generating adaptor tag nucleic sequence information based on the adaptor tag nucleic acid sequences.

Depletion Methods

In an embodiment, the present disclosure provides method for depleting a target nucleic acid sequence. In an embodiment, the method comprises (a) introducing to a sample solution, comprising a plurality of sample nucleic acid molecules each comprising a universal adaptor nucleic acid sequence comprising ribonucleotides, a capture primer nucleic acid molecule complementary or partially complementary to a target nucleic acid sequence of one or more sample nucleic acid molecules of the plurality of sample nucleic acid molecules; (b) enzymatically extending the capture primer nucleic acid molecule annealed to the target nucleic acid sequence of the one or more sample nucleic acid molecules; and (c) enzymatically cleaving double-stranded ribonucleic acid molecules of the sample nucleic acid molecules, to provide a depleted sample solution having a lower proportion of sample nucleic acid molecules comprising the target nucleic acid sequence than the sample solution.

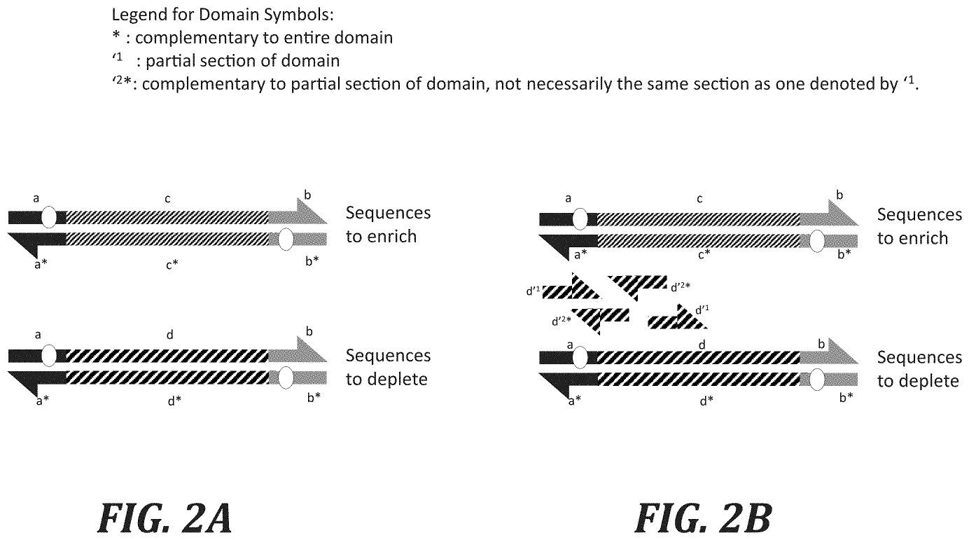

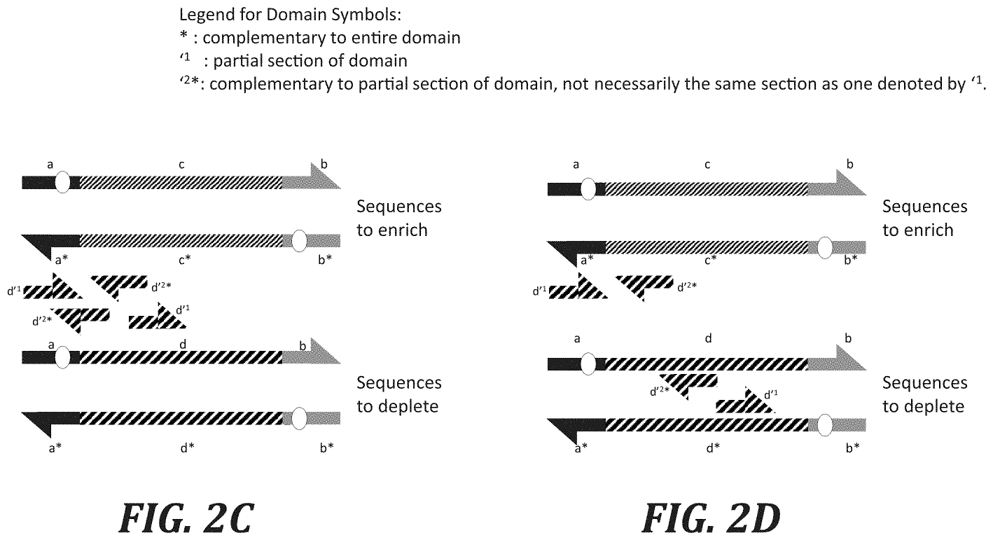

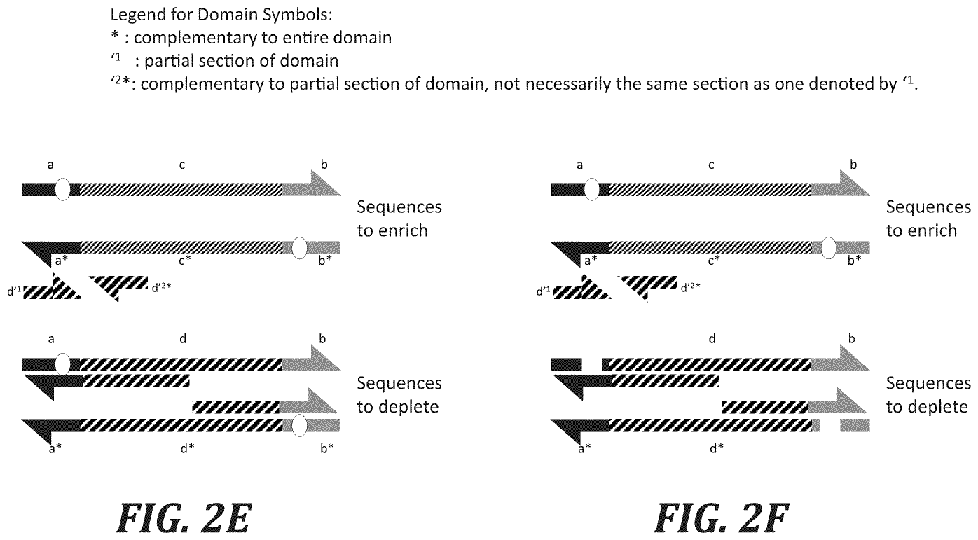

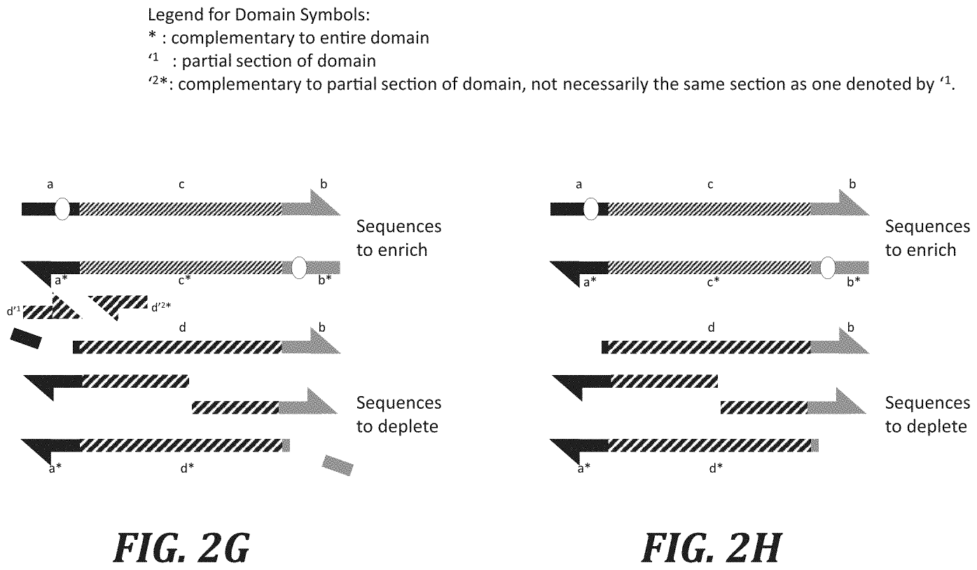

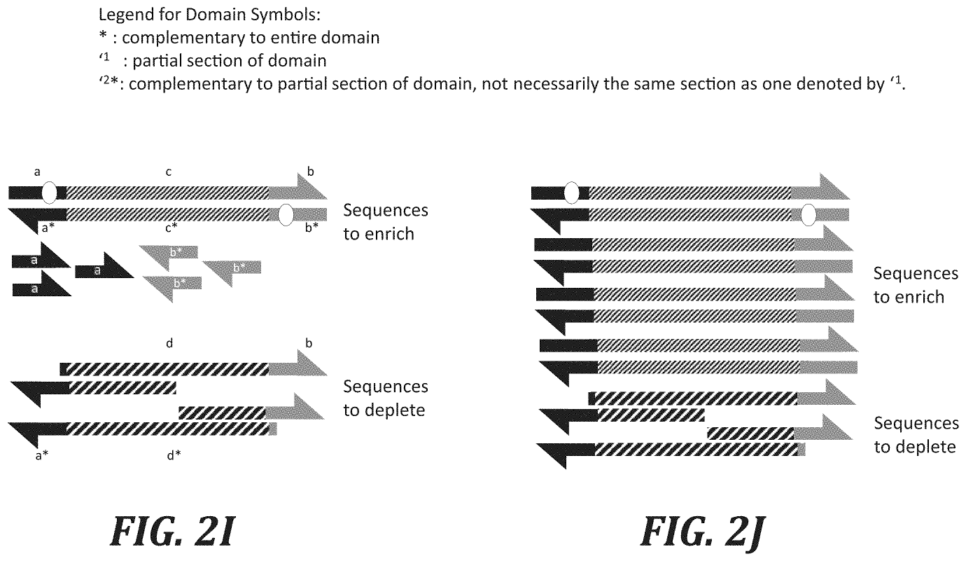

A method for depleting target nucleic acid sequences in accordance with an embodiment of the disclosure will now be described. In that regard, attention is directed to FIGS. 2A-2J, which schematically illustrates a method of depleting a target nucleic acid sequence, in accordance with an embodiment of the disclosure.

FIG. 2A schematically illustrates a sample solution including nucleic acid molecules to enrich and nucleic acid molecules to deplete. As shown, the sample solution includes a starting pool of nucleic acid molecules including a double-stranded nucleic acid molecule for enrichment and a double stranded nucleic acid molecule for depletion. The double-stranded sample nucleic acid molecule for enrichment is shown to include universal adaptor nucleic acid sequences a, a*, b, and b*, and target nucleic acid sequences c and c*. The double-stranded nucleic acid molecule for depletion is shown to include universal adaptor nucleic acid sequences a, a*, b, and b*, and target nucleic acid sequences d and d* different from the nucleic acid sequences c and c*. The universal adaptor nucleic acid sequences a, a*, b, and b*, on both the nucleic acid molecules for enrichment and for depletion, are shown to include a common feature, illustrated schematically here as an oval. As discussed further herein with respect to FIG. 2F, such a common feature is suitable for enzymatic degradation under certain conditions, such as where the universal adaptor nucleic acid sequence is double stranded.

The methods of the present disclosure are suitable to enrich a number of sample solutions comprising nucleic acid molecules. In an embodiment, the sample solution is selected from the group consisting of a WGS library, a WES library, ATAC-seq library, ChIP-seq library, WTS library, Bisulfite-seq library, RNA-seq library, single-cell RNA-seq library, DNA data storage library, or any other library with universal adapters on both ends. The mixture of DNA molecules can be previously amplified or unamplified, generated enzymatically or chemically synthesized. The universal adapters (domain a and domain b*) can include DNA and/or RNA nucleotides. As discussed further herein, at least one of the ribonucleotides may be a guanine. In an embodiment, the universal adaptor nucleic acid sequences present on all or substantially all nucleic acid molecules in the library.

In an embodiment, the sample solution includes double- or single-stranded sample nucleic acid molecules, such as from a WGS library, a WES library, ATAC-seq library, CHIP-seq library, WTS library, Bisulfite-seq library, RNA-seq library, and the like, containing 3′ modifications configured to prevent or limit self-annealing and extension. In an embodiment, such 3′ modifications include dideoxynucleotides (ddNTPs), inverted 3′dT, or nucleotide sequences that reduce binding energy (e.g. adenine, thymine, or uracil). In one embodiment, the starting sample solution includes double-stranded sample nucleic acid molecules, such as a WGS library, a WES library, ATAC-seq library, CHIP-seq library, WTS library, Bisulfite-seq library, RNA-seq library, and the like, generated by using PCR primers that contain polyT or polyA overhangs on the 5′ end.

In an embodiment, the universal adaptor nucleic acid sequences are added through PCR, transposition, reverse transcription, ligation, chemical synthesis, or other known methods to add adapters to DNA sequences, such as discussed further herein with respect to the kits of the present disclosure.

In an embodiment, the universal adaptor nucleic acid sequence includes a nucleic acid sequence adjacent to a 3′ end or a 5′ end that is configured not to bind to itself, such as in a hairpin configuration, thus avoiding self-priming. In an embodiment, the universal adaptor nucleic acid molecule includes a polyT sequence, a polyA sequence, or a combination thereof. See for example, FIGS. 6 and 7.

In an embodiment, nucleotides in the sample solution include ribonucleotides or deoxynucleotides. In an embodiment, such nucleotides include nucleotides selected from the group consisting of locked nucleic acids, peptide nucleic acids, 2′-O-methyl RNA, 2′-O:-methoxy ethyl RNA, phosphorothioate modified nucleic acids, and the like. Accordingly, in an embodiment, the degradation enzyme, such as RNase HII, is to be replaced with a degradation enzyme configured to selectively cleave the modified ribonucleotide or deoxynucleotide in a double-stranded conformation. In an embodiment, the sample nucleic acid molecules include methylated DNA and the degradation enzyme includes a restriction enzyme that specifically cleaves methylated (or hemimethylated) double stranded DNA.

As above, in an embodiment, the method includes introducing to the sample solution one or more capture primer nucleic acid molecule(s) complementary to or partially complementary to a target nucleic acid sequence of one or more sample nucleic acid molecules of the plurality of sample nucleic acid molecules. FIG. 2B schematically illustrates the sample solution of FIG. 2A further including capture primer nucleic acid molecules d′¹ and d′²*, in accordance with an embodiment of the disclosure. As shown, the capture primer nucleic acid molecules d′¹ and d′²* are complementary or partially complementary to a target nucleic acid sequences d* and d on the sample nucleic acid molecules for depletion, rather than the sample nucleic acid molecules for enrichment.

In an embodiment, the capture primer nucleic acid molecule is complementary to or partially complementary to the target nucleic acid sequence. In an embodiment, the capture primer nucleic acid molecule is partially complementary to the universal adaptor nucleic acid sequence. In an embodiment, the capture primer nucleic acid molecule comprises a number of bases that are not complementary to the universal adaptor nucleic acid sequence, such as in a range of 1 to 5. In an embodiment, the capture primer nucleic acid molecule is greater than or equal to 90% complementary to the universal adaptor sequence. Such partially complementary capture primer nucleic acid molecules are, nevertheless, configured to bind with target nucleic acid sequences, such as depending upon the annealing temperatures and/or other reaction conditions described herein.

In an embodiment, the method includes maintaining a temperature of the sample solution at or above a melting temperature of the plurality of sample nucleic acid molecules. FIG. 2C schematically illustrates the sample solution of FIG. 2B after melting the sample nucleic acid molecules to enrich and to deplete, in accordance with an embodiment of the disclosure. In an embodiment, the melting temperature is greater than or equal to 95° C. At melting temperature of the plurality of sample nucleic acid molecules the temperature of the sample solution is sufficient to completely or partially break Watson-Crick bonding between sample nucleic acid molecules, thereby increasing the number of single-stranded or partially single-stranded sample nucleic acid molecules in the sample solution. As shown, such melting exposes target nucleic acid sequences d and d*, as well as nucleic acid sequences c and c*, to bonding with other nucleic acid sequences, such as the capture primer nucleic acid molecules d′¹ and d′²*.

In an embodiment, the method includes maintaining the sample solution at about or below an annealing temperature of the capture primer nucleic acid molecule suitable to anneal the capture primer nucleic acid molecule to the target nucleic acid sequence. Such an annealing temperature is generally suitable to anneal at least a portion of the capture primer nucleic acid molecules to the target nucleic acid sequence. In an embodiment, the annealing temperature is in a range of about 50° C. to about 72° C. FIG. 2D schematically illustrates the sample solution of FIG. 2C after annealing capture primer nucleic acid molecules d′¹ and d′²*, to a target sequences d* and d of sample nucleic acid molecules to be depleted, in accordance with an embodiment of the disclosure. In the illustrated embodiment, capture primer nucleic acid molecules d′¹ and d′²* are bound to the target nucleic acid sequences d* and d of sample nucleic acid molecules to be depleted.

In an embodiment, the capture primer nucleic acid molecule is configured to be primarily single stranded at the annealing temperature. In this regard, the capture primer nucleic acid molecule is single stranded a majority of the time at the annealing temperature, and is, therefore, configured to bind to the target nucleic acid sequence a majority of the time. In an embodiment, the capture primer nucleic acid molecule is configured to be primarily at least partially double stranded at the annealing temperature. In this regard, the capture primer nucleic acid molecule is in a configuration suitable for binding to a target nucleic acid sequence less than a majority of the time at the annealing temperature. Thus, binding of such a double-stranded capture primer nucleic acid molecule to a target nucleic acid sequence is generally more selective than for single-stranded capture primer nucleic acid molecules.

In an embodiment, the capture primer nucleic acid molecule further comprises a second capture primer nucleic acid molecule complementary to or partially complementary to a first capture primer nucleic acid molecule. Such double-stranded capture primer nucleic acid molecules are generally double stranded at the annealing temperature and are, thus, less often configured to bind to a target nucleic acid sequence. In this regard, such double-stranded capture primer nucleic acid molecules are configured to bind more selectively to target nucleic acid sequences.

In an embodiment, the capture primer nucleic acid molecule is complementary to or partially complementary to a second target nucleic acid sequence of one or more second sample nucleic acid molecules of the plurality of sample nucleic acid molecules, wherein the second target nucleic acid sequence is different than the target nucleic acid sequence. In this regard, by maintaining the sample solution at or at about an annealing temperature of the capture primer nucleic acid molecule, the capture primer nucleic acid molecules may bind to various target nucleic acid sequences. As discussed further herein with respect to FIGS. 2E and 2F, sample nucleic acid molecules comprising various target sequences complementary to or partially complementary to the capture primer nucleic acid molecules may be enzymatically extended and marked for degradation.

In an embodiment, the capture primer nucleic acid molecule comprises a phosphorothioate linkage. In an embodiment, the phosphorothioate linkage is disposed between a base at a 3′ end of the capture primer nucleic acid molecule and a base immediately adjacent to the base at the 3′ end. Such phosphorothioate linkages are configured to resist 3′ exonuclease activity, such as those present in proof reading polymerases.

As above, the sample nucleic acid molecules include a universal adaptor nucleic acid sequence. In an embodiment, the universal adaptor nucleic acid sequence of the plurality of sample nucleic acid molecules comprises an adaptor tag nucleic acid sequence. In an embodiment, the adaptor tag nucleic acid sequence defines a unique nucleic acid sequence. Such unique sequence can be used to determine an origin of the sample nucleic acid molecules, such a cell, tissue, or suspension of origin, where such unique nucleic acid sequences have different sequences from another adaptor tag nucleic acid sequence used to tag sample nucleic acid molecules in other samples, such as in other cells, tissues, or suspensions of cell.

Such adaptor tag nucleic acid sequences are suitable for counting a number of nucleic acid molecules in a sample, such as through sequencing the sample solution. In an embodiment, each adaptor tag nucleic acid molecule includes a number of degenerate bases suitable for counting amplified sample nucleic acid molecules after a nucleic acid amplification reaction.

In an embodiment, an annealing temperature of the capture primer nucleic acid molecule and the second target nucleic acid sequence is relatively close to the annealing temperature of the capture primer nucleic acid molecule and the target nucleic acid sequence, such that by maintaining the sample solution at the annealing temperature of the capture primer nucleic acid molecule and the target nucleic acid sequence, at least some of the capture primer nucleic acid molecules bind to the second target nucleic acid sequence. Accordingly, in an embodiment, the capture primer nucleic acid molecule and the second target nucleic acid sequence have a second annealing temperature in a range of about 1° C. to about 5° C. of the annealing temperature.

In an embodiment, the sample solution is kept at temperatures that are near, but not necessarily precisely at, the annealing temperature. In this regard, the binding specificity of the capture primer nucleic acid molecules is varied, allowing the capture primer nucleic acid molecules to bind, for example, to a number of target nucleic acid sequences having relatively similar sequences, and thus depleting a number of different sample nucleic acid molecules. Accordingly, in an embodiment, maintaining the sample solution at about or below an annealing temperature of the capture primer nucleic acid molecule comprises maintaining the sample solution at a temperature within a range of about 1° C. to about 5° C. of the annealing temperature of the capture primer nucleic acid molecule.

As above, in an embodiment, the method includes enzymatically extending the capture primer nucleic acid molecule annealed to the target nucleic acid sequence of the one or more sample nucleic acid molecules. FIG. 2E schematically illustrates the sample solution of FIG. 2D after enzymatically extending the capture primer nucleic acid molecules d′¹ and d′²* annealed to the target sequences d* and d, in accordance with an embodiment of the disclosure. As shown, the capture primer nucleic acid molecules d′¹ and d′²* are annealed to the target sequences d* and d on the sample nucleic acid molecules to be depleted. As also shown, the nucleic acid sequence annealed to the target nucleic acid sequence d and d* are shown extended to also bind with the universal adaptor nucleic acid sequences a and b*. As discussed further herein, by binding to the universal adaptor nucleic acid sequences a and b*, the extended capture primer nucleic acid molecule activates enzymatic degradation of the double-stranded sample nucleic acid molecule.

The extension enzyme can include any enzyme configured to enzymatically extend the capture primer nucleic acid molecule annealed to another nucleic acid molecule. In an embodiment, the extension enzyme is selected from the group consisting of a polymerase, a reverse transcriptase, and combinations thereof.

In an embodiment, enzymatically extending the capture primer nucleic acid molecule comprises maintaining the sample solution at about an extension temperature of the extension enzyme suitable for enzymatic extension by the extension enzyme of the capture primer nucleic acid molecule annealed to the target nucleic acid sequence. Such an extension temperature may be the same as or different from the annealing temperature. In an embodiment, the annealing temperature is in a range of about 50° C. to about 72° C.

As above, the methods of the present embodiment include enzymatically cleaving double-stranded ribonucleic acid molecules of the sample nucleic acid molecules. FIG. 2F schematically illustrates the sample solution of FIG. 2E after enzymatically degrading double-stranded nucleic acid molecules, in accordance with an embodiment of the disclosure. In the illustrated embodiment, the universal adaptor nucleic acid sequences a and b* bound to the enzymatically extended capture primer nucleic acid molecules are degraded. In this regard, the ovals of the capture primer nucleic acid molecules are shown to be degraded. As discussed further herein with respect to FIG. 2F, such degradation can include cleaving or degrading a backbone of the universal adaptor nucleic acid sequence of the double-stranded sample nucleic acid molecules.

In the illustrated embodiment, the degradation enzyme is shown to have enzymatically degraded a portion of the double-stranded nucleic acid molecule including the universal adaptor sequences a and b*, including the targeted portion of the universal adaptor nucleic acid sequence (illustrated here as an oval). Sample nucleic acid molecules including the target nucleic acid sequences d and d* have enzymatically degraded universal adaptor sequences a and b*. This is in contrast to the single-stranded sample nucleic acid, which includes the nucleic acid sequence c and c*, which have intact universal adaptor sequences. In this regard, the single-stranded sample nucleic acid is shown to have an intact universal adaptor nucleic acid sequence.

Enzymatic degradation of the double-stranded sample nucleic acid molecules can include a number of forms of degradation configured, for example, to make the degraded sample nucleic acid unsuitable for nucleic acid amplification reactions, such as those including the universal adaptor nucleic acid molecules. In an embodiment, enzymatically degrading the double-stranded sample nucleic acid molecules includes cleaving a backbone of the universal adaptor nucleic acid molecule of the double-stranded sample nucleic acid molecules. In an embodiment, enzymatically cleaving the double-stranded sample nucleic acid molecules includes degrading a portion of the universal adaptor nucleic acid sequence disposed on the double-stranded sample nucleic acid molecules. In an embodiment, enzymatically cleaving the double-stranded sample nucleic acid molecules includes cleaving a backbone of the universal adaptor nucleic acid sequence of the double-stranded sample nucleic acid molecules. In an embodiment, enzymatically cleaving the double-stranded sample nucleic acid molecules includes digesting a portion of the universal adaptor nucleic acid sequence of the double-stranded sample nucleic acid molecules.

In an embodiment, enzymatically degrading double-stranded sample nucleic acid molecules comprises maintaining the temperature of the sample solution at a degradation temperature of the degradation enzyme. In an embodiment, the degradation temperature is below the annealing temperature. In an embodiment, the degradation temperature is below the extension temperature. In an embodiment, the degradation temperature is less than or equal to about 60° C.

In an embodiment, the degradation temperature is an active temperature of the degradation enzyme. Accordingly, by maintaining the sample solution at or at about the degradation, the degradation enzyme is active, such as active in degrading double-stranded nucleic acid molecules. In an embodiment, the degradation enzyme is inactive at a temperature chosen from the extension temperature, the melting temperature, the annealing temperature, and combinations thereof. In this regard, the degradation enzyme does not or does not substantially enzymatically degrade double-stranded nucleic acid molecules in the sample solution, such as before enzymatic extension of annealed capture primer nucleic acid molecules annealed to the target nucleic acid sequences.

In an embodiment, the degradation enzyme is active at the degradation temperature after being inactive at a temperature above the degradation temperature, such as the extension temperature. In this regard, in an embodiment, the degradation enzyme is configured to preferentially or selectively degrade sample nucleic acid molecules, such as double-stranded sample nucleic acid molecules, after having been inactive at a temperature above the degradation temperature. Without wishing to be bound by theory, it is believed that the degradation enzyme is inactive above the active temperature, such as when the degradation enzyme takes on an inactive conformation, and that the degradation further becomes active when the degradation enzyme assumes an active configuration when the temperature of the sample solution is maintained in an active range.

As above, in an embodiment, the degradation enzyme is configured to enzymatically degrade double-stranded nucleic acid molecules, such as double-stranded sample nucleic acid molecules. In an embodiment, the degradation enzyme is not a restriction endonuclease. In an embodiment, the degradation enzyme is a ribonuclease. In an embodiment, the degradation enzyme is an endonuclease. In an embodiment, the endonuclease is an endoribonuclease. In an embodiment, the endoribonuclease is selected from the group consisting of Rnase HII, RNase H, Rnase III, and combinations thereof.

In an embodiment, the degradation enzyme is Rnase HII. In an embodiment, the degradation enzyme is according to SEQ ID NO. 16. In an embodiment, the degradation has a sequence homology to SEQ ID NO. 16 greater to 90%, greater than 95%, or greater than 99%.

In an embodiment, the degradation enzyme is Rnase H. In an embodiment, the degradation enzyme is according to SEQ ID NO. 17. In an embodiment, the degradation has a sequence homology to SEQ ID NO. 17 greater to 90%, greater than 95%, or greater than 99%.

In an embodiment, the degradation enzyme is Rnase III. In an embodiment, the degradation enzyme is according to SEQ ID NO. 18. In an embodiment, the degradation has a sequence homology to SEQ ID NO. 18 greater to 90%, greater than 95%, or greater than 99%.

In an embodiment, the method of the present disclosure includes repeating enzymatically extending the capture primer nucleic acid molecule and enzymatically degrading double-stranded sample nucleic acid molecules. By repeating enzymatic extension and enzymatic degradation, the extension enzyme, capture primer nucleic acid molecules, and degradation enzyme can be used one or more additional times to selectively degrade sample nucleic acid molecules that include a target nucleic acid sequence, such as target sequences d and d*. As above, in an embodiment, such degradation includes degrading the universal adaptor nucleic acid sequence, which can be later used in a nucleic acid amplification reaction. As discussed further herein with respect to FIGS. 2I and 2J, nucleic acid sequences that include intact universal adaptor nucleic acid sequences are preferentially enriched. Accordingly, by enzymatically degrading additional sample nucleic acid sequences that have target nucleic acid sequences, sample nucleic acid molecules that do not have the target nucleic acid sequences a configured not to take part in such selective or preferential enrichment.

In an embodiment, the method further includes maintaining the temperature of the sample solution at or above a melting temperature of the plurality of sample nucleic acid molecules and the capture primer nucleic acid molecule, such as after enzymatically extending the capture primer nucleic acid molecule and enzymatically degrading double-stranded sample nucleic acid molecules. In this regard, the sample solution including sample nucleic acid molecules having enzymatically degraded or intact universal adaptor nucleic acid sequences are single stranded and, thus configured to later bind with capture primer nucleic acid molecules. FIG. 2G schematically illustrates melting the nucleic acid molecules of the sample solution of FIG. 2F, in accordance with an embodiment of the disclosure.

In an embodiment, the method of the present disclosure includes purifying the plurality of sample nucleic acid molecules in the depleted sample solution. FIG. 2H schematically illustrates the sample solution of FIG. 2G after removing the capture primer nucleic acid molecules d′¹ and d′²*, in accordance with an embodiment of the disclosure. Such purification can include, for example, purification with SPRI beads and the like. In an embodiment, purifying the plurality of sample nucleic acid molecules in the depleted sample solution comprises removing reagents chosen from capture primer nucleic acid molecules, enzymes, and combinations thereof from the depleted sample solution. Such purification of the sample solution can simplify sequencing data based on the sample solution, such as by reducing the number of nucleic acid molecules present in the sample solution and, thereby, decreasing an amount of sequencing data based on the sample solution, particularly reducing an amount of sequencing data not related to target nucleic acid sequences.

In an embodiment, the method of the present disclosure includes amplifying sample nucleic acid molecules after enzymatic degradation of double-stranded nucleic acid molecules. Accordingly, in an embodiment the method includes introducing a plurality of amplification primer nucleic acid molecules to the depleted sample solution. In an embodiment, the amplification primer nucleic acid molecules of the plurality of amplification primer nucleic acid molecules are complementary to the universal adaptor nucleic acid sequence. FIG. 2I schematically illustrates the sample solution of FIG. 2H further including polymerase chain reaction (PCR) primers a and b*. As shown, the PCR primers are complementary to the universal adaptor sequences a* and b of the nucleic acid molecules in the sample solution, in accordance with an embodiment of the disclosure.

In an embodiment, the method includes performing a nucleic acid amplification reaction on the plurality of sample nucleic acid molecules in the depleted sample solution with the plurality of amplification primer nucleic acid molecules to provide an amplified depleted sample solution. FIG. 2J schematically illustrates the sample solution of FIG. 2I after PCR amplification of the nucleic acid molecules of the sample solution, in accordance with an embodiment of the disclosure. As shown, the sample solution includes a greater proportion of sample nucleic acid molecules including nucleic acid sequences c and c* than samples nucleic acid molecules including the target nucleic acid sequences d and d*.

As discussed above and shown in FIG. 2J, because at least some of the universal adaptor nucleic acid sequences of the sample nucleic acid molecules are degraded, these degraded sample nucleic acid molecules will not participate in the nucleic acid amplification reaction, and thus the amplified depleted sample solution will contain a lower proportion of such sample nucleic acid molecules. In this regard, in an embodiment, performing the nucleic acid amplification reaction on the plurality of sample nucleic acid molecules in the depleted sample solution does not or does not substantially amplify sample nucleic acid molecules that have been degraded by the degradation enzyme. Accordingly, the amplified depleted sample solution comprises a greater proportion of nucleic acid molecules that include sequences c or c* than d or d* compared to the original sample solution shown in FIG. 2A.

In an embodiment, the method includes performing one or more enzymatic reactions on the amplified depleted sample to solution to prepare the depleted sample solution for sequencing, such as a next-generation sample preparation. Accordingly, in an embodiment, the method of the present disclosure includes performing a reaction on the amplified depleted sample solution chosen from a nucleic acid fragmentation reaction, enzymatic end repair, A tailing, adaptor ligation, polymerase chain reaction, and combinations thereof.

In an embodiment, the method of the present disclosure includes sequencing nucleic acid molecules in the depleted sample solution. In an embodiment, sequencing nucleic acid molecules in the depleted sample solution comprises generating sample nucleic acid information based upon the plurality of sample nucleic acid molecules in the depleted sample solution. As above, in certain embodiment, the universal adaptor nucleic acid molecules include an adaptor tag nucleic acid molecule. In an embodiment, sequencing nucleic acid molecules in the depleted sample solution comprises generating adaptor tag nucleic sequence information based on the adaptor tag nucleic acid sequences.

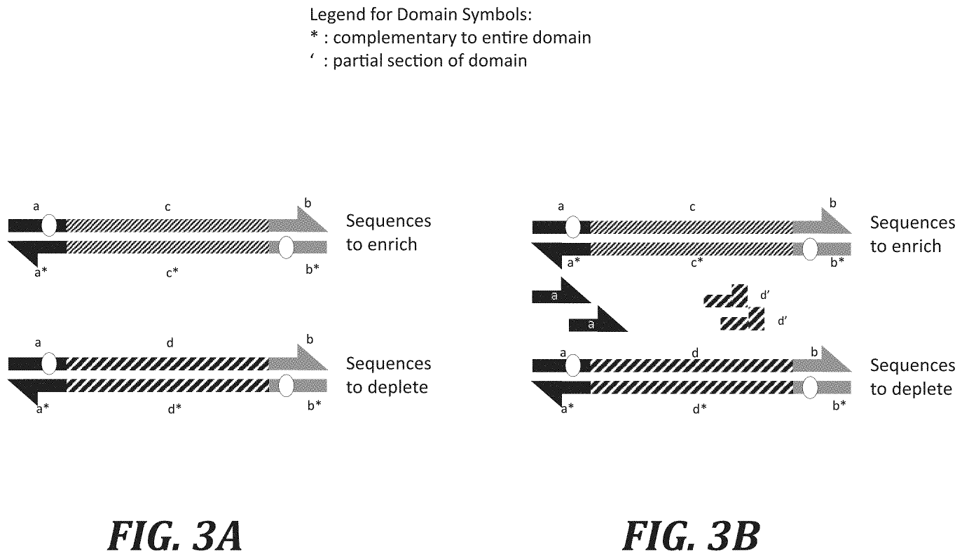

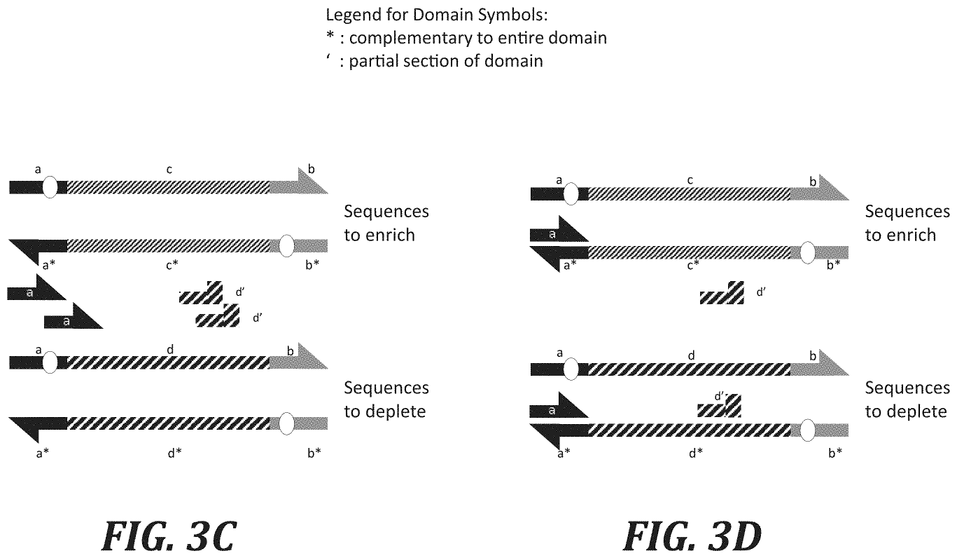

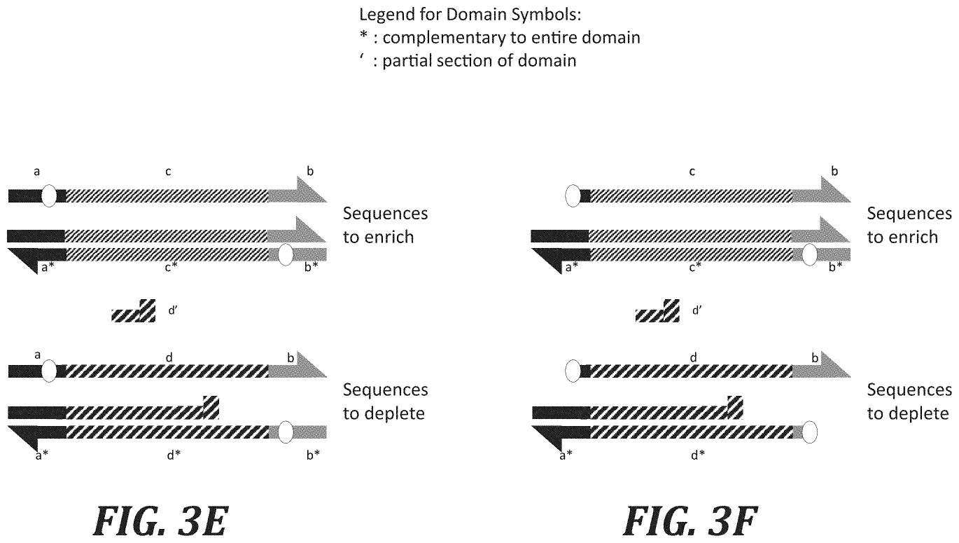

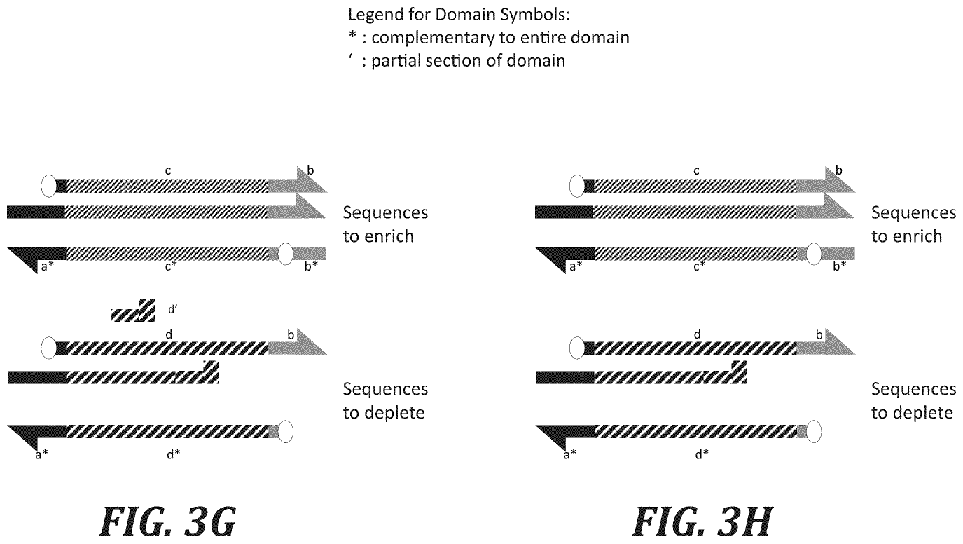

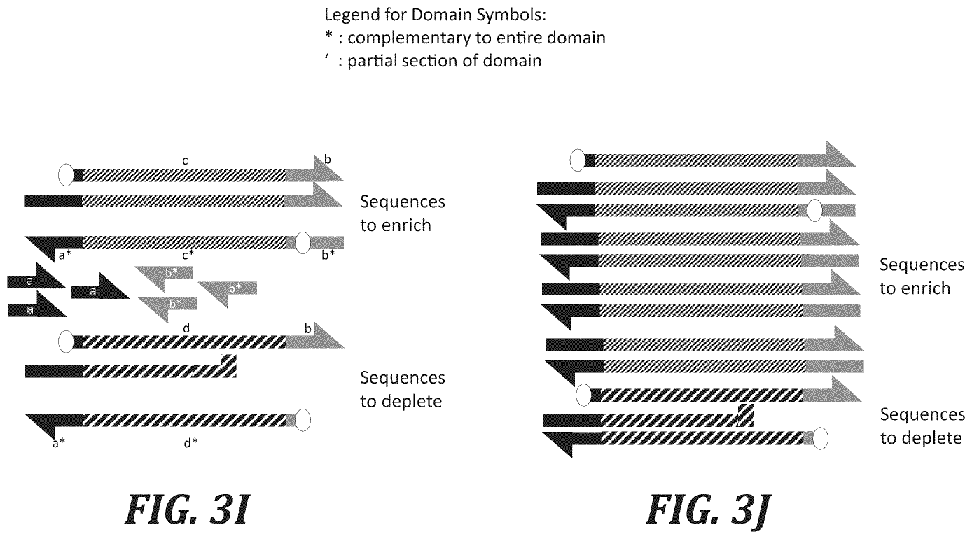

In an embodiment, the capture primer nucleic acid molecule is a blocked capture primer nucleic acid molecule. In that regard, attention is directed to FIGS. 3A-3J, in which a method in accordance with an embodiment of the disclosure is illustrated. FIGS. 3A-3D are analogous to FIGS. 1A-1D, described elsewhere herein, except that the capture primer nucleic acid molecules include capture primer nucleic acid molecule d′, which is a blocked capture primer nucleic acid molecule. In that regard, in an embodiment, the blocked capture primer nucleic acid molecule d′ is configured to block enzymatic extension at a 3′ end of the blocked capture primer nucleic acid molecule d′ by an extension enzyme. As shown, the sample solution further includes a non-blocked capture primer nucleic acid molecule a.

In an embodiment, the blocked capture primer nucleic acid molecule includes an inverted nucleic acid. In an embodiment, the blocked capture primer nucleic acid molecule includes one or more overhanging adenines or thymines at a 3′ end.

As shown in FIG. 3E, enzymatic extension where the blocked capture primer nucleic acid molecule is annealed to target nucleic acid sequence d*, the extension enzyme is unable to extend past the blocked capture primer nucleic acid molecule, whereas on other sample nucleic acid molecules, the extension enzyme has successfully extended across the whole molecule, such as the sample nucleic acid molecule for enrichment, which does not include the target nucleic acid sequence d*.

As shown in FIG. 3F, the degradation enzyme has enzymatically degraded the single-stranded universal adaptor molecule of the sample nucleic acid to be depleted. In this regard, as the sample solution is subsequently melted (FIG. 3G), purified (FIG. 3H), and amplified (FIGS. 3I and 3J), molecules including the target nucleic acid sequence d* are depleted and the sample solution is shown to have a higher proportion of sample nucleic acid molecules having sequences c and c* than the target nucleic acid sequences d and d*. The sample solution is, thus, depleted of sample nucleic acid molecules having the target nucleic acid sequence.

While blocked capture primer nucleic acid molecules are shown to deplete sample nucleic acid molecules in conjunction with degradation enzymes configured to degrade single-stranded nucleic acid molecules, blocked capture primer nucleic acid molecules can be used in conjunction with degradation enzymes configured to degrade double-stranded sample nucleic acid molecules to enrich for sample nucleic acid molecules having a target nucleic acid sequence complementary to the blocked capture primer nucleic molecules, in accordance with an embodiment of the disclosure.

### Kits

In another aspect, the present disclosure provides kits including reagents for enriching and/or depleting target nucleic target nucleic acid sequences, such as target nucleic acid sequences present in complex sample solutions comprising nucleic acid molecules that do not include the target nucleic acid sequence.

Enrichment Kits

In an embodiment, the present disclosure provides a kit for enriching sample nucleic acid molecules including a target nucleic acid sequence. In an embodiment, the kit includes a capture primer nucleic acid molecule complementary to or partially complementary to a target sequence; and a degradation enzyme configured to degrade a single-stranded nucleic acid molecule.

As above, the kit includes a capture primer nucleic acid molecule. In an embodiment, the capture primer nucleic acid molecule perfectly complementary to a target nucleic acid sequence. In an embodiment, the capture primer nucleic acid molecule is partially complementary to one or more target nucleic acid molecules. As discussed further herein, the capture primer nucleic acid molecules can be at least partially complementary to a number of target nucleic acid sequences, and, thus, the kits of the present disclosure are configured to enrich sample nucleic acid molecules having a number of different target nucleic acid sequences, depending upon the reaction conditions in which they are deployed.

As discussed further herein with respect to FIG. 1D, the capture primer nucleic acid molecules can be single stranded, at least partially double stranded, or double stranded, such as at an annealing temperature between the capture primer nucleic acid molecule and its target nucleic acid sequence.

In an embodiment, the capture primer nucleic acid molecule comprises a phosphorothioate linkage. In an embodiment, the phosphorothioate linkage is disposed between a base at a 3′ end of the capture primer nucleic acid molecule and a base immediately adjacent to the base at the 3′ end. Such phosphorothioate linkages are configured to resist 3′ exonuclease activity, such as those present in proof reading polymerases.

In an embodiment, the kit further includes a plurality of universal adaptor nucleic acid molecules configured to couple to a sample nucleic acid molecule. As discussed further herein with respect to the methods of the present disclosure, the universal adaptor nucleic acid molecules are suitable for use in a nucleic acid amplification reaction.

In an embodiment, the universal adaptor nucleic acid molecule comprises a riboguanine, such as where the degradation enzyme is Rnase T1. In an embodiment, the universal adaptor nucleic acid molecule comprises a ribocytosine, a ribouracil, or combinations thereof, such as where the degradation enzyme is Rnase A.

In an embodiment, the universal adaptor nucleic acid molecule includes a nucleic acid sequence adjacent to a 3′ end or a 5′ end that is configured not to bind to itself, such as in a hairpin configuration, thus avoiding self-priming. In an embodiment, the universal adaptor nucleic acid molecule includes a polyT sequence, a polyA sequence, or a combination thereof.

In an embodiment, the kit further comprises reagents for coupling the universal adaptor nucleic acid molecule to a sample nucleic acid molecule. In an embodiment, the kit comprises selected from the group consisting of a transposase loaded with an oligonucleotide comprising a universal adaptor nucleic acid molecule; a restriction endonuclease, an oligonucleotide or oligonucleotide complex comprising a universal adaptor nucleic acid molecule, an oligonucleotide or oligonucleotide complex comprising a T7 promoter, an antibody or antibody fragment against a transcription factor, and combinations thereof.

The kits of the present embodiment include a degradation enzyme. In an embodiment, the degradation enzyme configured to degrade a single-stranded nucleic acid molecule. In an embodiment, the degradation enzyme is configured to degrade single-stranded nucleic acid molecules comprising the universal adaptor nucleic acid molecule. In an embodiment, the degradation enzyme is a ribonuclease. In an embodiment, the degradation enzyme is an endonuclease. In an embodiment, the endonuclease is an endoribonuclease. In an embodiment, the endoribonuclease is selected from the group consisting of Rnase T1, Rnase A, and combinations thereof.

In an embodiment, the degradation enzyme is Rnase T1. In an embodiment, the degradation enzyme is according to SEQ ID NO. 14. In an embodiment, the degradation has a sequence homology to SEQ ID NO. 14 greater to 90%, greater than 95%, or greater than 99%. In an embodiment, the universal adaptor nucleic acid sequence comprises a riboguanine. In an embodiment, the universal adaptor nucleic acid sequence comprises a plurality of riboguanines. Rnase T1 selectively degrades single-stranded riboguanines, and, accordingly, where the universal adaptor nucleic acid sequence includes one or more riboguanines, the Rnase T1 degradation enzyme is configured to degrade the universal adaptor nucleic acid sequence, such as when the sample solution is maintained at an active temperature of Rnase T1.

In an embodiment, the degradation enzyme is Rnase A. In an embodiment, the degradation enzyme is according to SEQ ID NO. 15. In an embodiment, the degradation has a sequence homology to SEQ ID NO. 15 greater to 90%, greater than 95%, or greater than 99%. In an embodiment, the universal adaptor nucleic acid sequence comprises bases selected from the group consisting of a ribocytosine, a ribouracil, and combinations thereof. In an embodiment, the universal adaptor nucleic acid sequence comprises a plurality of ribocytosines, a plurality of ribouracils, and combinations thereof. Rnase A selectively degrades single-stranded ribocytosines and ribouracils (such as at salt concentrations above 300 mM), and accordingly, where the universal adaptor nucleic acid sequences includes one or more ribocytosines and/or ribouracils, the Rnase A degradation enzyme is configured to degrade the universal adaptor nucleic acid sequence, such as when the sample solution is maintained at an active temperature of Rnase A.

In an embodiment, the degradation enzyme is inactive in degrading single-stranded nucleic acid molecules above an active temperature range; and active in degrading single-stranded nucleic acid molecules within the active temperature range after having been inactive. As discussed further herein, in an embodiment, the degradation enzyme is inactive at elevated temperatures, such as at an enzymatic extension temperature, but is active once the temperature of a sample solution is lowered after having been elevated.

In an embodiment, the kit further comprises an extension enzyme configured to extend a capture primer nucleic acid molecule annealed to the target nucleic acid sequence. In an embodiment, the extension enzyme is selected from the group consisting of a polymerase, a reverse transcriptase, and combinations thereof.

In an embodiment, the kit further comprises instructions for enriching a target nucleic acid sequence, such as in a sample comprising sample nucleic acid molecules. In an embodiment, the kit comprises instructions for enriching sample nucleic acid molecules including a target nucleic acid sequence. In an embodiment, the instructions comprise instructions comprising: (a) introducing to a sample solution, comprising a plurality of sample nucleic acid molecules each comprising a universal adaptor nucleic acid sequence, a capture primer nucleic acid molecule complementary to or partially complementary to a target nucleic acid sequence of one or more sample nucleic acid molecules of the plurality of sample nucleic acid molecules; (b) enzymatically extending the capture primer nucleic acid molecule annealed to the target nucleic acid sequence of the one or more sample nucleic acid molecules; and (c) enzymatically degrading single-stranded sample nucleic acid molecules, to provide an enriched sample solution having a higher proportion of sample nucleic acid molecules comprising the target nucleic acid sequence than the sample solution. In an embodiment, the instructions further comprise repeating steps (b) and (c) one or more times on the enriched sample solution. In an embodiment, the instructions further comprise maintaining the temperature of the sample solution at or above a melting temperature of the plurality of sample nucleic acid molecules and the capture primer nucleic acid molecule.

In an embodiment, the instructions for enzymatically extending the capture primer nucleic acid molecule comprise: maintaining a temperature of the sample solution at or above a melting temperature of the plurality of sample nucleic acid molecules; introducing to the sample solution an extension enzyme configured to extend the capture primer nucleic acid molecule annealed to the target nucleic acid sequence; and maintaining the sample solution at about or below an annealing temperature of the capture primer nucleic acid molecule suitable to anneal the capture primer nucleic acid molecule to the target nucleic acid sequence; and maintaining the sample solution at about an extension temperature of the extension enzyme suitable for enzymatic extension by the extension enzyme of the capture primer nucleic acid molecule annealed to the target nucleic acid sequence.

In an embodiment, the instructions for enzymatically degrading single-stranded sample nucleic acid molecules comprise: introducing to the sample solution a degradation enzyme configured to degrade a single-stranded nucleic acid molecule comprising the universal adaptor nucleic acid sequence; and maintaining the temperature of the sample solution at a degradation temperature of the degradation enzyme.

In an embodiment, the instructions further comprise instructions for coupling universal adaptor molecules to samples nucleic acid molecules in a sample solution.

Depletion Kits

In an embodiment, the present disclosure provides a kit for depleting a sample nucleic acid molecule including a target nucleic acid sequence. In an embodiment, the kit comprising a capture primer nucleic acid molecule complementary to or partially complementary to a target sequence; and a degradation enzyme configured to degrade a double-stranded nucleic acid molecule.

As above, the kit includes a capture primer nucleic acid molecule. In an embodiment, the capture primer nucleic acid molecule perfectly complementary to a target nucleic acid sequence. In an embodiment, the capture primer nucleic acid molecule is partially complementary to one or more target nucleic acid molecules. As discussed further herein, the capture primer nucleic acid molecules can be at least partially complementary to a number of target nucleic acid sequences, and, thus, the kits of the present disclosure are configured to enrich sample nucleic acid molecules having a number of different target nucleic acid sequences, depending upon the reaction conditions in which they are deployed.

As discussed further herein with respect to FIG. 2D, the capture primer nucleic acid molecules can be single stranded, at least partially double stranded, or double stranded, such as at an annealing temperature between the capture primer nucleic acid molecule and its target nucleic acid sequence.

In an embodiment, the capture primer nucleic acid molecule comprises a phosphorothioate linkage. In an embodiment, the phosphorothioate linkage is disposed between a base at a 3′ end of the capture primer nucleic acid molecule and a base immediately adjacent to the base at the 3′ end. Such phosphorothioate linkages are configured to resist 3′ exonuclease activity, such as those present in proof reading polymerases.

In an embodiment, the kit further includes a plurality of universal adaptor nucleic acid molecules configured to couple to a sample nucleic acid molecule. As discussed further herein with respect to the methods of the present disclosure, the universal adaptor nucleic acid molecules are suitable for use in a nucleic acid amplification reaction.

In an embodiment, the universal adaptor nucleic acid molecule includes a nucleic acid sequence adjacent to a 3′ end or a 5′ end that is configured not to bind to itself, such as in a hairpin configuration, thus avoiding self-priming. In an embodiment, the universal adaptor nucleic acid molecule includes a polyT sequence, a polyA sequence, or a combination thereof.

In an embodiment, the kit further comprises reagents for coupling the universal adaptor nucleic acid molecule to a sample nucleic acid molecule. In an embodiment, the kit comprises selected from the group consisting of a transposase loaded with an oligonucleotide comprising a universal adaptor nucleic acid molecule; a restriction endonuclease, an oligonucleotide or oligonucleotide complex comprising a universal adaptor nucleic acid molecule, an oligonucleotide or oligonucleotide complex comprising a T7 promoter, an antibody or antibody fragment against a transcription factor, and combinations thereof.

The kits of the present embodiment include a degradation enzyme. In an embodiment, the degradation enzyme is configured to cleave double-stranded nucleic acid molecules comprising the universal adaptor nucleic acid molecule. In an embodiment, the degradation enzyme is configured to degrade double-stranded nucleic acid molecules comprising the universal adaptor nucleic acid molecule. In an embodiment, the degradation enzyme is a ribonuclease. In an embodiment, the degradation enzyme is an endonuclease. In an embodiment, the endonuclease is an endoribonuclease. In an embodiment, the degradation enzyme is not a restriction endonuclease. In an embodiment, the degradation enzyme is a ribonuclease. In an embodiment, the degradation enzyme is an endonuclease. In an embodiment, the endonuclease is an endoribonuclease. In an embodiment, the endoribonuclease is selected from the group consisting of Rnase HII, RNase H, Rnase III, and combinations thereof.

In an embodiment, the degradation enzyme is Rnase HII. In an embodiment, the degradation enzyme is according to SEQ ID NO. 16. In an embodiment, the degradation has a sequence homology to SEQ ID NO. 16 greater to 90%, greater than 95%, or greater than 99%.

In an embodiment, the degradation enzyme is Rnase H. In an embodiment, the degradation enzyme is according to SEQ ID NO. 17. In an embodiment, the degradation has a sequence homology to SEQ ID NO. 17 greater to 90%, greater than 95%, or greater than 99%.

In an embodiment, the degradation enzyme is Rnase III. In an embodiment, the degradation enzyme is according to SEQ ID NO. 18. In an embodiment, the degradation has a sequence homology to SEQ ID NO. 18 greater to 90%, greater than 95%, or greater than 99%.

In an embodiment, the kit further comprises an extension enzyme configured to extend a capture primer nucleic acid molecule annealed to the target nucleic acid sequence. In an embodiment, the extension enzyme is selected from the group consisting of a polymerase, a reverse transcriptase, and combinations thereof.

In an embodiment, the kit further comprises instructions for depleting a target nucleic acid sequence, such as in a sample comprising sample nucleic acid molecules. In an embodiment, the instructions comprise instructions for performing the depletion methods of the present disclosure. In an embodiment, the instructions comprise (a) introducing to a sample solution, comprising a plurality of sample nucleic acid molecules each comprising a universal adaptor nucleic acid sequence comprising ribonucleotides, a capture primer nucleic acid molecule complementary or partially complementary to a target nucleic acid sequence of one or more sample nucleic acid molecules of the plurality of sample nucleic acid molecules; (b) enzymatically extending the capture primer nucleic acid molecule annealed to the target nucleic acid sequence of the one or more sample nucleic acid molecules; and (c) enzymatically cleaving double-stranded ribonucleic acid molecules of the sample nucleic acid molecules, to provide a depleted sample solution having a lower proportion of sample nucleic acid molecules comprising the target nucleic acid sequence than the sample solution. In an embodiment, the instructions further comprise repeating steps (b) and (c) one or more times on the enriched sample solution. In an embodiment, the instructions further comprise maintaining the temperature of the sample solution at or above a melting temperature of the plurality of sample nucleic acid molecules and the capture primer nucleic acid molecule.

In an embodiment, the instructions for enzymatically extending the capture primer nucleic acid molecule comprise maintaining a temperature of the sample solution at or above a melting temperature of the plurality of sample nucleic acid molecules; introducing to the sample solution an extension enzyme configured to extend the capture primer nucleic acid molecule annealed to the target nucleic acid sequence; and maintaining the sample solution at about or below an annealing temperature of the capture primer nucleic acid molecule suitable to anneal the capture primer nucleic acid molecule to the target nucleic acid sequence; and maintaining the sample solution at about an extension temperature of the extension enzyme suitable for enzymatic extension by the extension enzyme of the capture primer nucleic acid molecule annealed to the target nucleic acid sequence.

In an embodiment, the instructions for enzymatically cleaving double-stranded sample nucleic acid molecules comprise introducing to the sample solution a degradation enzyme configured to cleave a double-stranded nucleic acid molecule comprising the universal adaptor nucleic acid sequence; and maintaining the temperature of the sample solution at a degradation temperature of the degradation enzyme.

In an embodiment, the instructions further comprise: introducing a plurality of amplification primer nucleic acid molecules to the depleted sample solution, wherein amplification primer nucleic acid molecules of the plurality of amplification primer nucleic acid molecules are complementary to the universal adaptor nucleic acid sequence; and performing a nucleic acid amplification reaction on the plurality of sample nucleic acid molecules in the depleted sample solution with the plurality of amplification primer nucleic acid molecules.

In an embodiment, the instructions further comprise instructions for coupling universal adaptor molecules to samples nucleic acid molecules in a sample solution.

### EXAMPLES

### Example 1: Example Results of Enrichment Strategy Using Selection Probes

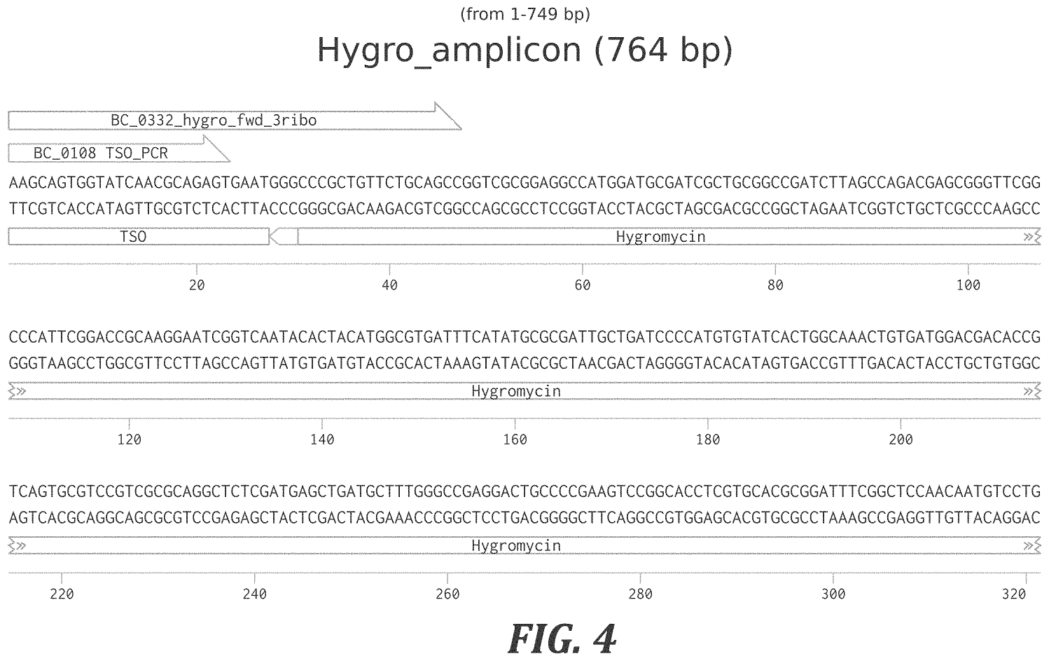

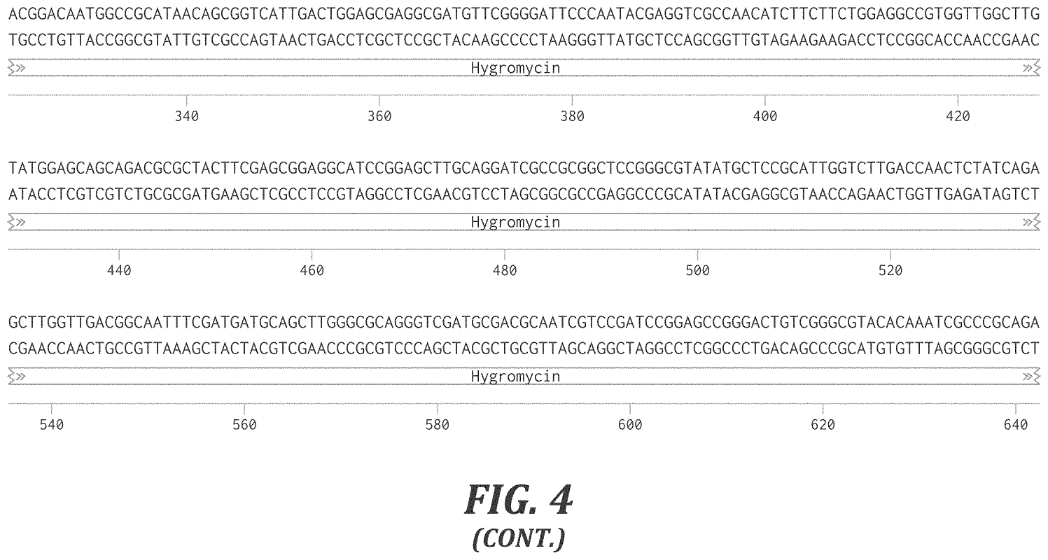

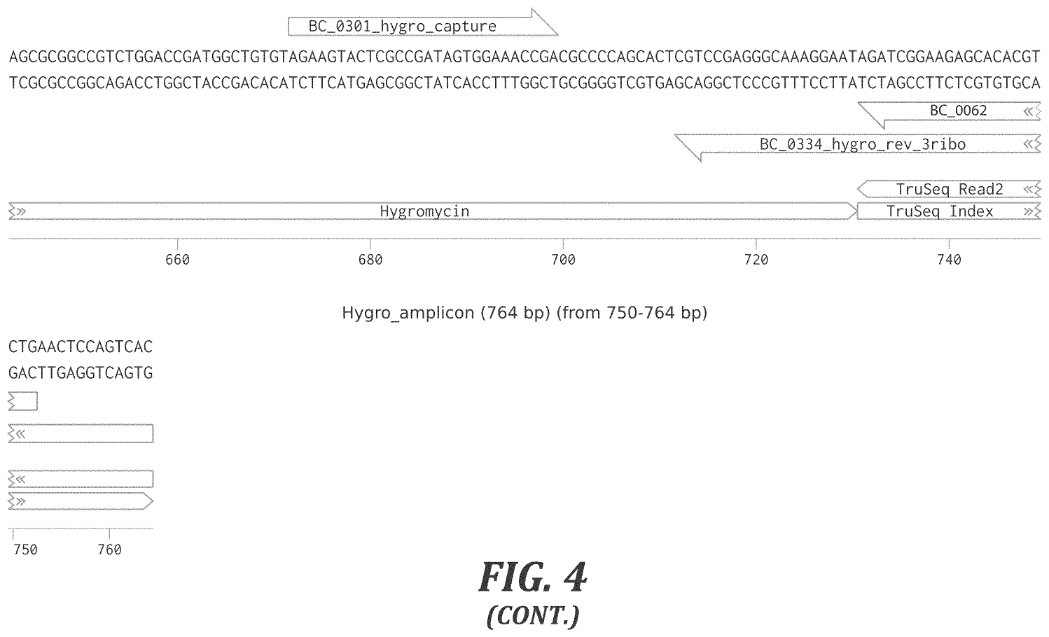

Two different amplicons of different length with universal adapters were generated by amplifying sequences from a plasmid (AmpR: 421 bp and Hygro: 774 bp). Primers BC_0328 and BC_0330 were used to generate the AmpR amplicon (FIG. 6). Primers BC_0332 and BC_0334 were used to generate the Hygro amplicon (FIG. 4).

Equal amounts of both amplicons (0.2 ng each) were added to 20 uL reactions.

To enrich for the AmpR amplicon, we used the following mix, where BC_306_amp_capture is an oligonucleotide that is complementary to the AmpR amplicon, but not the Hygro amplicon.

TABLE 1

| Component | 20 uL reaction |
|---|---|
| 10x Standard Taq buf | 2 |
| 10 mM DNTPs | 0.4 |
| Amp + Hygro amplicon mix (0.4 ng total) | 1 |
| Taq DNA Polymerase | 0.1 |
| BC_0306_amp_capture | 0.4 |
| 10x Diluted RNase T1 | 1 |
| Nuclease-free H2O | 15.1 |
| Total Volume | 20 |

To enrich for the Hygro amplicon, we used the following mix, where BC_301_hygro_capture is an oligonucleotide that is complementary to the Hygro amplicon, but not the AmpR amplicon.

TABLE 2

| Component | 20 uL reaction |
|---|---|
| 10x Standard Taq buf | 2 |
| 10 mM DNTPs | 0.4 |
| Amp + Hygro amplicon mix (0.4 ng total) | 1 |
| Taq DNA Polymerase | 0.1 |
| BC_0301_hygro_capture | 0.4 |
| 10x Diluted RNase T1 | 1 |
| Nuclease-free H2O | 15.1 |
| Total Volume | 20 |

Samples were then cycled with the following conditions:

Thermocycle samples with the following protocol for 1 or 3 cycles:
1. 95° C.-30 s
2. 58′C—20 s
3. 68° C.-20 s
4. 37° C. or 42 C or 50 C—15 min

Then samples were immediately put on ice

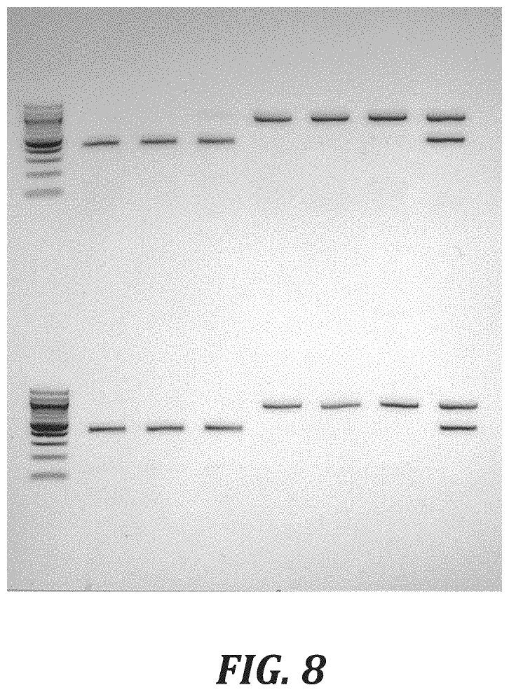

2 uL of each reaction was then added to a 25 uL qPCR reaction with universal primers. Once reactions began to plateau, they were removed from qPCR and run on a 1.25% agarose gel. The results are shown in FIG. 8.

Left to Right in Top Row:
1. 100 base-pair ladder (New England Biolabs)
2. Amp capture, 1 cycle, step 4 at 37° C.
3. Amp capture, 1 cycle, step 4 at 42° C.
4. Amp capture, 1 cycle, step 4 at 50° C.
5. Hygro capture, 1 cycle, step 4 at 37° C.
6. Hygro capture, 1 cycle, step 4 at 42° C.
7. Hygro capture, 1 cycle, step 4 at 50° C.
8. Control: DNA only

Left to Right in Bottom Row:
9. 100 base-pair ladder (New England Biolabs)
10. Amp capture, 3 cycles, step 4 at 37° C.
11. Amp capture, 3 cycles, step 4 at 42° C.
12. Amp capture, 3 cycles, step 4 at 50° C.
13. Hygro capture, 3 cycles, step 4 at 37° C.
14. Hygro capture, 3 cycles, step 4 at 42° C.
15. Hygro capture, 3 cycles, step 4 at 50° C.
16. Control: DNA only

Oligonucleotide Sequences:

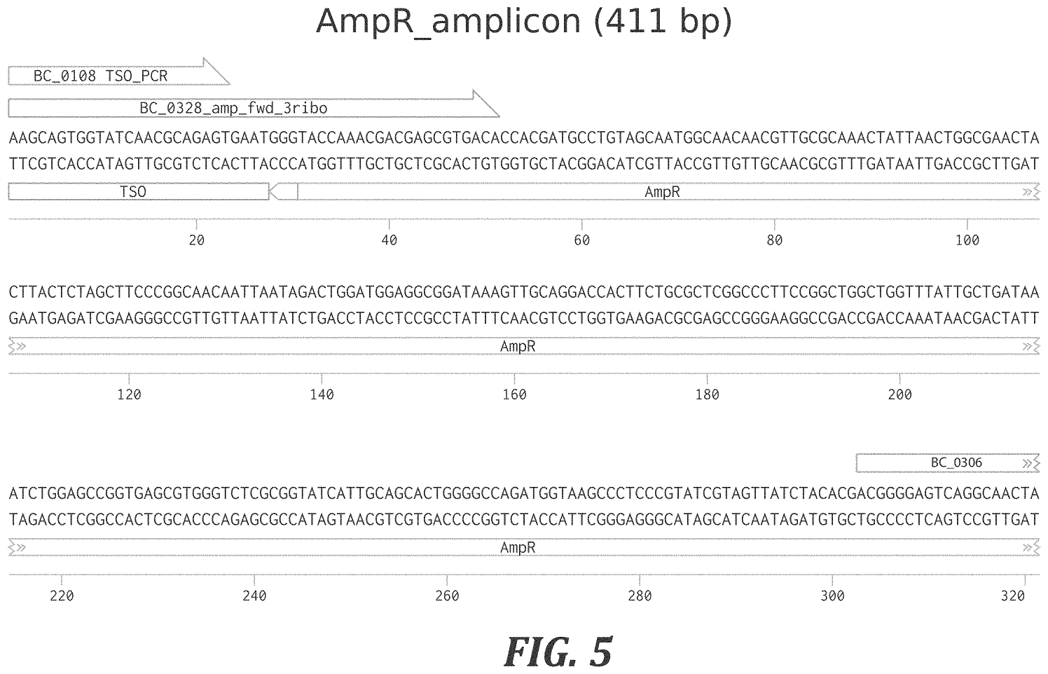

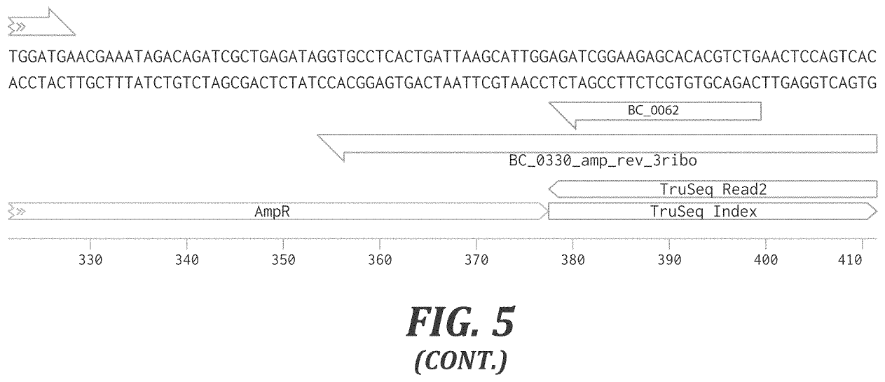

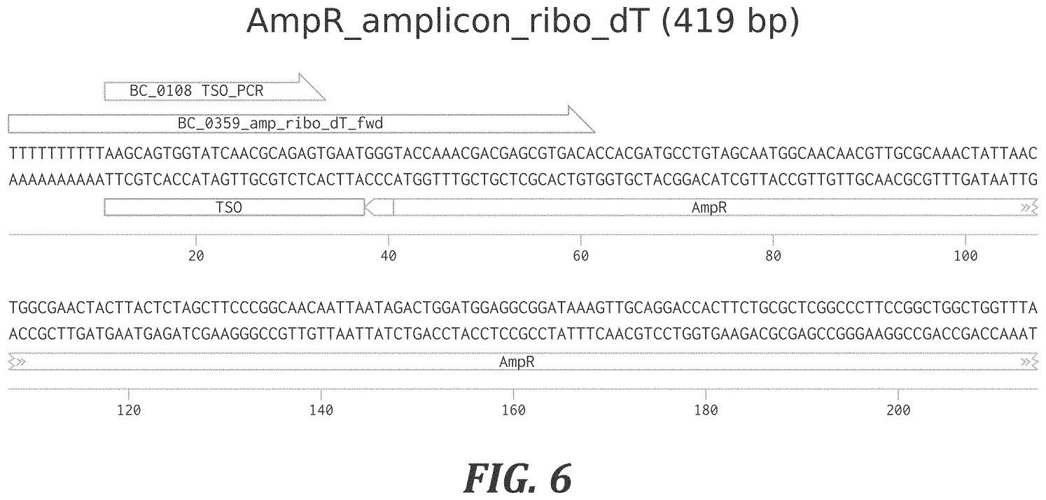

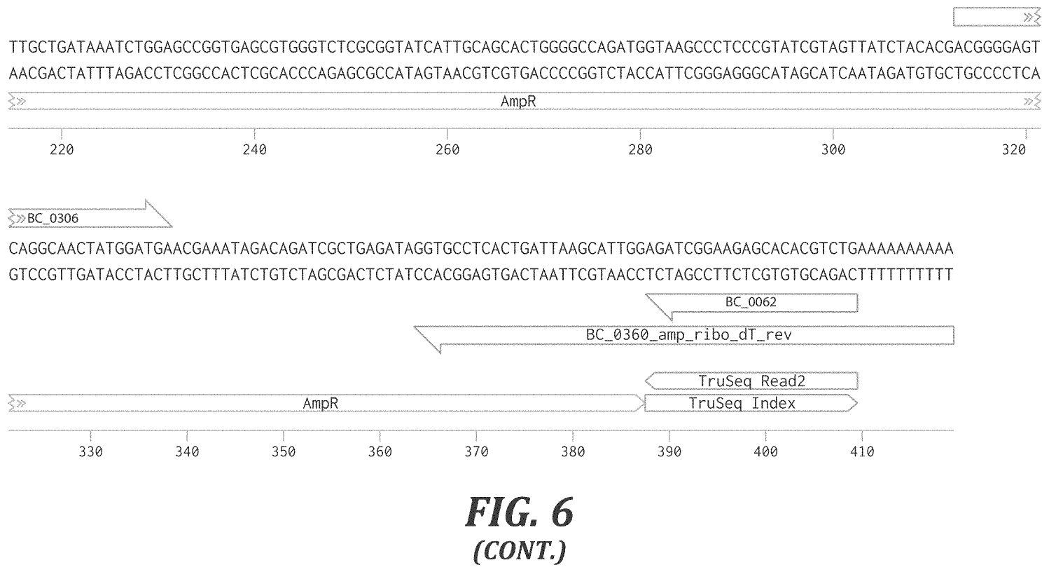

See FIG. 5: AmpR_amplicon-sequence.pdf
See FIG. 4: Hygro_amplicon-sequence.pdf
BC_0108_TSO_PCR
(SEQ ID NO. 12)
AAGCAGTGGTATCAACGCAGAGT
BC_0062_Primer_Bind
(SEQ ID NO. 13)
CAGACGTGTGCTCTTCCGATCT
BC_0328_amp_fwd_3ribo
(SEQ ID NO. 1)
AAGCAGTGGTATCAACrGCArGAGTrGAATGGGTACCAAACGACGAGCGT
GACA
BC_0330_amp_rev_3ribo
(SEQ ID NO. 2)
GTGACTGGAGTTCAGACrGTGTrGCTCTTCCrGATCTCCAATGCTTAATC
AGTGAGGCACC
BC_0306_Amp_capture
(SEQ ID NO. 19)
ACGGGGAGTCAGGCAACTATGGATGA
BC_0359_amp_ribo_dT_fwd
(SEQ ID NO. 20)
TTTTTTTTTTAAGCAGTGGTATCAACrGCArGAGTrGAATGGGTACCAAA
CGACGAGCGTGACA
BC_0360_amp_ribo_dT_rev
(SEQ ID NO. 21)
TTTTTTTTTTCAGACrGTGTrGCTCTTCCrGATCTCCAATGCTTAATCAGT
GAGGCACC
BC_0332_hygro_fwd_3ribo
(SEQ ID NO. 3)
AAGCAGTGGTATCAACrGCArGAGTrGAATGGGCCCGCTGTTCTGCAGCC
BC_0334_hygro_rev_3ribo
(SEQ ID NO. 4)
GTGACTGGAGTTCAGACrGTGTrGCTCTTCCrGATCTATTCCTTTGCCCT
CGGACG
BC_0301_hygro_capture
(SEQ ID NO. 22)
AGAAGTACTCGCCGATAGTGGAAACCGA

The results from the gel image in FIG. 8 show enrichment of the desired target molecules across a variety of conditions. Lanes 2-4 and 10-12 show enrichment of the AmpR molecules. Lanes 5-7 and 13-15 show enrichment of the Hygro molecules. Enrichment occurs across a range of temperatures for the degradation step (37° C.-50° C.). The gel also shows that multiple cycles of melting nucleic acids, annealing capture primers, extending capture primers, and degrading single stranded riboguanines can lead to equivalent or higher fold enrichment than a single cycle (compare lanes 10-12 to lanes 4-6 and lanes 13-15 to lanes 5-7).

### Example 2

In this example, single-cell RNA-sequencing libraries (from expanded primary T-cells) were enriched for specific sequences matching parts of the following genes:

ACTB
(ATGGCCCAGTCCTCTCCCAA, SEQ ID NO. 5),
GAPDH
(AGGAGTAAGACCCCTGGACCAC, SEQ ID NO. 6),
TRAC
(AGAACCCTGACCCTGCCG, SEQ ID NO. 7),
TRBC1
(CTGAAAAACGTGTTCCCACCCGAG, SEQ ID NO. 8),
and
TRBC2
(ACCTGAACAAGGTGTTCCCACC, SEQ ID NO. 9)).

TRAC corresponds to the constant region of the T cell receptor alpha chain, while TRBC1 and TRCB2 correspond to two possible constant regions of the T cell receptor beta chain. T cell receptor alpha and beta chains are generated by VJ and VDJ recombination leading to a very high diversity of possible sequences for each. However, by enriching nucleic acid sequences containing part of the TRAC sequence, it is possible to enrich all or nearly all nucleic acid sequences coding for the T cell receptor alpha chain, and similarly by enriching nucleic acid sequences containing part of either the TRBC1 or TRBC2, it is possible to enrich all or nearly all nucleic acid sequences coding for the T cell receptor beta chain.

A single-cell RNA-sequencing library of amplified cDNA was generated according the published SPLiT-seq method. 1 ng of amplified cDNA was reamplified for 11 cycles of PCR using primers BC_385 and BC_386 to introduce riboguanosines into to each 5′ end of the double stranded DNA molecules. The resulting PCR products were purified with SPRI beads (Kapa Pure Beads) using a 2:1 ratio of beads to PCR product according to the manufacturer's instructions. The concentration of the resulting purified PCR product was measured using the Qubit dsDNA HS Assay Kit.

In total 12 different variations of enrichment were compared. 3 different polymerase mixes were tested, two different polymerase extension times were tested, and two concentrations of Rnase T1 were tested (3×2×2=12 combinatorial variations).

Variation 1 (Hot start Taq in 1× Standard Taq Buffer, 30 s polymerase extension, 100 u Rnase T1)

Variation 2 (Hot start Taq in 1× Standard Taq Buffer, 120 s polymerase extension, 100 u Rnase T1) Variation 3 (Hot start Taq in 1× Standard Taq Buffer, 30 s polymerase extension, 20 u Rnase T1)

Variation 4 (Hot start Taq in 1× Standard Taq Buffer, 120 s polymerase extension, 20 u Rnase T1)

Variation 5 (OneTaq Hot start in 1× OneTaq Standard Reaction Buffer, 30 s polymerase extension, 100 u Rnase T1)

Variation 6 (OneTaq Hot start in 1× OneTaq Standard Reaction Buffer, 120 s polymerase extension, 100 u Rnase T1)

Variation 7 (OneTaq Hot start in 1× OneTaq Standard Reaction Buffer, 30 s polymerase extension, 20 u Rnase T1) Variation 8 (OneTaq Hot start in 1× OneTaq Standard Reaction Buffer, 120 s polymerase extension, 20 u Rnase T1)

Variation 9 (Deep Vent Exo—in 1× ThermoPol Reaction Buffer, 30 s polymerase extension, 100 u Rnase T1)

Variation 10 (Deep Vent Exo—in 1× ThermoPol Reaction Buffer, 120 s polymerase extension, 100 u Rnase T1)

Variation 11 (Deep Vent Exo—in 1× ThermoPol Reaction Buffer, 30 s polymerase extension, 20 u Rnase T1)

Variation 12 (Deep Vent Exo—in 1× ThermoPol Reaction Buffer, 120 s polymerase extension, 20 u Rnase T1)

Each reaction was prepared with:

(2 uL 10× Standard Taq Buffer/4 uL OneTaq Standard Reaction Buffer/2 uL ThermoPol Reaction Buffer), 1.6 uL 2.5 mM dNTPs, (0.1 uL HotStart Taq Polymerase/0.1 uL OneTaq® Hot Start DNA Polymerase/0.1 uL Deep Vent® (exo-) DNA Polymerase), 1 uL of pooled capture primers (10 uM total, 2 uM each), (11.3/13.3 uL water), 1 uL of amplified cDNA (from PCR using BC_385 and BC_386), and 1 uL of Rnase T1 (diluted to 100 u/uL or 20 u/uL). Primers BC_0344_ACTB_probe (SEQ ID NO. 5), BC_0343_GAPDH_probe (SEQ ID NO. 6), BC_0391_TRAC_probe (SEQ ID NO. 7), BC_0392_TRBC1_probe (SEQ ID NO. 8), BC_0393_TRBC2_probe (SEQ ID NO. 9) were used as the pooled capture primers.

Variations 1, 3, 5, 7 were cycled as follows:

a. 95 C for 30 s, b. 95 C for 30 s, c. 53 C for 20 s, d. 68 C for 30 s, e. 37 C for 15 min, f. repeat steps b-e 2 additional cycles (3 including first cycle).

Variations 2, 4, 6, 8 were cycled as follows:

a. 95 C for 30 s, b. 95 C for 30 s, c. 53 C for 20 s, d. 68 C for 2 min, e. 37 C for 15 min, f. repeat steps b-e 2 additional cycles (3 including first cycle).

Variations 9 and 11 were cycled as follows:

a. 95 C for 30 s, b. 95 C for 30 s, c. 55 C for 20 s, d. 72 C for 30 s, e. 37 C for 15 min, f. repeat steps b-e 2 additional cycles (3 including first cycle).

Variations 10 and 12 were cycled as follows:

a. 95 C for 30 s, b. 95 C for 30 s, c. 55 C for 20 s, d. 72 C for 2 min, e. 37 C for 15 min, f. repeat steps b-e 2 additional cycles (3 including first cycle).

All 12 reactions were then purified using a single sided SPRI cleanup (Kapa Pure Beads) according to the manufacturer's instructions (2× ratio of beads to PCR product). Each of the 12 purified reactions were then amplified with PCR using primers BC_0062 (SEQ ID NO. 12) and BC_0108 TSO_PCR (SEQ ID NO. 12). The amplified PCR products were then prepared for next generation sequencing on an Illumina sequencer by fragmentation, end-repair (including A-tailing), adapter ligation, and PCR with primers to add indexed Illumina adapters (P7 and P5).

The original amplified cDNA library (which did not undergo any enrichment) was also prepared for next generation sequencing using the same methods (fragmentation, end-repair (including A-tailing), adapter ligation, and PCR with primers to add indexed Illumina adapters (P7 and P5)).

All 13 libraries (12 variations of enrichments and original non-enriched library) were sequenced together on an Illumina NextSeq. The resulting libraries were demultiplexed according to indices added during the final PCR.

The fold-change enrichment for each of the 12 enrichment variations relative to the non-enriched library was then calculated for each of the 5 sequences that were intended to be enriched:

ACTB
(ATGGCCCAGTCCTCTCCCAA, SEQ ID NO. 5),
GAPDH
(AGGAGTAAGACCCCTGGACCAC, SEQ ID NO. 6),
TRAC
(AGAACCCTGACCCTGCCG, SEQ ID NO. 7),
TRBC1
(CTGAAAAACGTGTTCCCACCCGAG, SEQ ID NO. 8),
and
TRBC2
(ACCTGAACAAGGTGTTCCCACC, SEQ ID NO. 9)).

TABLE 3
Fold-change Enrichment:

| Variation | Rnase T1 | | Polymerase | Poly_ext_time | | ACTB | GAPDH | TRAC | TRBC1 | TRBC2 |
|---|---|---|---|---|---|---|---|---|---|---|
| 1 | 100 | U | Hotstart_Taq | 30 | s | 1.61 | 1.35 | 3.31 | 2.12 | 1.85 |
| 2 | 100 | U | Hotstart_Taq | 120 | s | 2.61 | 2.01 | 4.41 | 2.81 | 2.58 |
| 3 | 20 | U | Hotstart_Taq | 30 | s | 7.75 | 6.91 | 10.59 | 8.04 | 8.09 |
| 4 | 20 | U | Hotstart_Taq | 120 | s | 12.77 | 12.50 | 15.76 | 15.03 | 15.78 |
| 5 | 100 | U | OneTaq_hotstart | 30 | s | 4.35 | 4.12 | 10.23 | 8.58 | 7.74 |
| 6 | 100 | U | OneTaq_hotstart | 120 | s | 5.03 | 5.26 | 10.86 | 9.87 | 8.97 |
| 7 | 20 | U | OneTaq_hotstart | 30 | s | 6.51 | 7.49 | 9.49 | 9.11 | 9.08 |
| 8 | 20 | U | OneTaq_hotstart | 120 | s | 8.88 | 11.31 | 12.44 | 13.48 | 13.26 |
| 9 | 100 | U | Deep_Vent_exo | 30 | s | 14.90 | 11.38 | 20.42 | 16.56 | 17.01 |
| 10 | 100 | U | Deep_Vent_exo | 120 | s | 16.10 | 18.19 | 20.39 | 20.35 | 20.87 |
| 11 | 20 | U | Deep_Vent_exo | 30 | s | 13.67 | 8.89 | 16.80 | 13.99 | 14.31 |
| 12 | 20 | U | Deep_Vent_exo | 120 | s | 14.36 | 14.77 | 17.33 | 18.28 | 18.07 |

The results in Table 3 show enrichment of the desired target molecules across a variety of conditions. For each of the five target sequences, nucleic acids containing the given sequence are enriched across different experimental conditions. The concentration of Rnase T1, type of polymerase, and polymerase extension time can be adjusted resulting in different fold enrichment of the target sequences.

While illustrative embodiments have been illustrated and described, it will be appreciated that various changes can be made therein without departing from the spirit and scope of the invention.

1. A method for enriching a target nucleic acid sequence, the method comprising:
(a) introducing to a sample solution, comprising a plurality of sample nucleic acid molecules each comprising a universal adaptor nucleic acid sequence, a capture primer nucleic acid molecule complementary to or partially complementary to a target nucleic acid sequence of one or more sample nucleic acid molecules of the plurality of sample nucleic acid molecules;
(b) enzymatically extending the capture primer nucleic acid molecule annealed to the target nucleic acid sequence of the one or more sample nucleic acid molecules; and
(c) enzymatically degrading single-stranded sample nucleic acid molecules, to provide an enriched sample solution having a higher proportion of sample nucleic acid molecules comprising the target nucleic acid sequence than the sample solution.

2. The method of claim 1, wherein enzymatically extending the capture primer nucleic acid molecule comprises:
maintaining a temperature of the sample solution at or above a melting temperature of the plurality of sample nucleic acid molecules;
introducing to the sample solution an extension enzyme configured to extend the capture primer nucleic acid molecule annealed to the target nucleic acid sequence;
maintaining the sample solution at about or below an annealing temperature of the capture primer nucleic acid molecule suitable to anneal the capture primer nucleic acid molecule to the target nucleic acid sequence; and
maintaining the sample solution at about an extension temperature of the extension enzyme suitable for enzymatic extension by the extension enzyme of the capture primer nucleic acid molecule annealed to the target nucleic acid sequence.

3. The method of claim 2, wherein the extension enzyme is selected from the group consisting of a polymerase, a reverse transcriptase, and combinations thereof.

4. The method of claim 1, wherein enzymatically degrading single-stranded sample nucleic acid molecules comprises:
introducing to the sample solution a degradation enzyme configured to degrade a single-stranded nucleic acid molecule comprising the universal adaptor nucleic acid sequence; and
maintaining the temperature of the sample solution at a degradation temperature of the degradation enzyme.

5. The method of claim 4, wherein the degradation enzyme is introduced to the sample solution after enzymatically extending the capture primer nucleic acid molecule.

6. The method of claim 4, wherein the degradation enzyme is introduced to the sample solution before enzymatically extending the capture primer nucleic acid molecule.

7. The method of claim 4, wherein the degradation temperature is below the annealing temperature.

8. The method of claim 4, wherein the degradation temperature is below the extension temperature.

9. The method of claim 4, wherein the degradation temperature is an active temperature of the degradation enzyme.

10. The method of claim 4, wherein the degradation enzyme is a ribonuclease.

11. The method of claim 4, wherein the degradation enzyme is an endonuclease.

12. The method of claim 11, wherein the endonuclease is an endoribonuclease.

13. The method of claim 12, wherein the endoribonuclease is selected from the group consisting of Rnase T1, Rnase A, and combinations thereof.

14. The method of claim 4, wherein the degradation enzyme is Rnase T1, and wherein the universal adaptor nucleic acid sequence comprises a riboguanine.

15. The method of claim 4, wherein the degradation enzyme is Rnase A, and wherein the universal adaptor nucleic acid sequence comprises base selected from the group consisting of a ribocytosine, a ribouracil, and combinations thereof.

16. The method of claim 4, wherein the degradation enzyme is inactive at the extension temperature.

17. The method of claim 4, wherein the degradation enzyme is active at the degradation temperature after being inactive at a temperature above the degradation temperature.

18. The method of claim 1, wherein enzymatically degrading the single-stranded sample nucleic acid molecules includes degrading a portion of the universal adaptor nucleic acid sequence disposed on the single-stranded sample nucleic acid molecules.

19. The method of claim 1, wherein enzymatically degrading the single-stranded sample nucleic acid molecules includes cleaving a backbone of the universal adaptor nucleic acid molecule of the single-stranded sample nucleic acid molecules.

20. The method of claim 1, wherein enzymatically degrading the single-stranded sample nucleic acid molecules includes digesting a portion of the universal adaptor nucleic acid molecule of the single-stranded sample nucleic acid molecules.

21. The method of claim 1, wherein the capture primer nucleic acid molecule is complementary to or partially complementary to a second target nucleic acid sequence of one or more second sample nucleic acid molecules of the plurality of sample nucleic acid molecules, wherein the second target nucleic acid sequence is different than the target nucleic acid sequence.

22. The method of claim 2, wherein maintaining the sample solution at about or below an annealing temperature of the capture primer nucleic acid molecule comprises maintaining the sample solution at a temperature within a range of about 1° C. to about 5° C. of the annealing temperature of the capture primer nucleic acid molecule.

23. The method of claim 2, wherein the capture primer nucleic acid molecule and the second target nucleic acid sequence have a second annealing temperature in a range of about 1° C. to about 5° C. of the annealing temperature.

24. The method of claim 2, further comprising repeating steps (b) and (c) one or more times on the enriched sample solution.

25. The method of claim 24, further comprising maintaining the temperature of the sample solution at or above a melting temperature of the plurality of sample nucleic acid molecules and the capture primer nucleic acid molecule.

26. The method of claim 1, further comprising:
introducing a plurality of amplification primer nucleic acid molecules to the enriched sample solution, wherein amplification primer nucleic acid molecules of the plurality of amplification primer nucleic acid molecules are complementary to the universal adaptor nucleic acid sequence; and
performing a nucleic acid amplification reaction on the plurality of sample nucleic acid molecules in the enriched sample solution with the plurality of amplification primer nucleic acid molecules to provide an amplified enriched sample solution.

27. The method of claim 26, wherein performing the nucleic acid amplification reaction on the plurality of sample nucleic acid molecules in the enriched sample solution does not or does not substantially amplify sample nucleic acid molecules that have been degraded by the degradation enzyme.

28. The method of claim 26, further comprising performing a reaction on the amplified enriched sample solution chosen from a nucleic acid fragmentation reaction, enzymatic end repair, A tailing, adaptor ligation, polymerase chain reaction, and combinations thereof.

29. The method of claim 1, further comprising purifying the plurality of sample nucleic acid molecules in the enriched sample solution.

30. The method of claim 29, wherein purifying the plurality of sample nucleic acid molecules in the enriched sample solution comprises removing reagents chosen from capture primer nucleic acid molecules, enzymes, and combinations thereof from the enriched sample solution.

31. The method of claim 1, further comprising sequencing nucleic acid molecules in the enriched sample solution.

32. The method of claim 1, wherein the universal adaptor nucleic acid sequence of the plurality of sample nucleic acid molecules comprises an adaptor tag nucleic acid sequence.

33. The method of claim 32, wherein the adaptor tag nucleic acid sequence defines a unique nucleic acid sequence.

34. The method of claim 31, wherein sequencing nucleic acid molecules in the enriched sample solution comprises generating sample nucleic acid information based upon the plurality of sample nucleic acid molecules in the enriched sample solution.

35. The method of claim 34, wherein sequencing nucleic acid molecules in the enriched sample solution comprises generating adaptor tag nucleic sequence information based on the adaptor tag nucleic acid sequences.

36. The method of claim 1, wherein the capture primer nucleic acid molecule comprises a phosphorothioate linkage.

37. The method of claim 36, wherein the phosphorothioate linkage is disposed between a base at a 3′ end of the capture primer nucleic acid molecule and a base immediately adjacent to the base at the 3′ end.

38. The method of claim 1, wherein the capture primer nucleic acid molecule is configured to be primarily single stranded at the annealing temperature.

39. The method of claim 1, wherein the capture primer nucleic acid molecule is configured to be primarily at least partially double stranded at the annealing temperature.

40. The method of claim 1, wherein the capture primer nucleic acid molecule is partially complementary to the target nucleic acid sequence, and wherein the capture primer nucleic acid molecule comprises a number of bases that are not complementary to the universal adaptor nucleic acid sequence in a range of 1 to 5.

41. The method of claim 1, wherein the capture primer nucleic acid molecule is partially complementary to the target nucleic acid sequence, and wherein the capture primer nucleic acid molecule is greater than or equal to 90% complementary to the universal adaptor sequence.

42. The method of claim 1, wherein the capture primer nucleic acid further comprises a second capture primer nucleic acid molecule complementary to or partially complementary to a first capture primer nucleic acid molecule.

43. A kit comprising:
a capture primer nucleic acid molecule complementary to or partially complementary to a target sequence; and
a degradation enzyme configured to degrade a single-stranded nucleic acid molecule.

44-64. (canceled)

65. A method for depleting a target nucleic acid sequence, the method comprising:
(a) introducing to a sample solution, comprising a plurality of sample nucleic acid molecules each comprising a universal adaptor nucleic acid sequence comprising ribonucleotides, a capture primer nucleic acid molecule complementary or partially complementary to a target nucleic acid sequence of one or more sample nucleic acid molecules of the plurality of sample nucleic acid molecules;
(b) enzymatically extending the capture primer nucleic acid molecule annealed to the target nucleic acid sequence of the one or more sample nucleic acid molecules; and
(c) enzymatically cleaving double-stranded ribonucleic acid molecules of the sample nucleic acid molecules, to provide a depleted sample solution having a lower proportion of sample nucleic acid molecules comprising the target nucleic acid sequence than the sample solution.

66-104. (canceled)

105. A kit comprising:
a capture primer nucleic acid molecule complementary to or partially complementary to a target sequence; and
a degradation enzyme configured to degrade a double-stranded nucleic acid molecule.

106-124. (canceled)


SEQUENCE LISTING (22 sequences)

SEQ ID NO: 1 - DNA, 51 bp, Artificial sequence
  Synthetic
  misc_feature (1)..(51): DNA/RNA Hybrid
  misc_feature (17)..(17): G at position 17 is RNA
  misc_feature (20)..(20): G at position 20 is RNA
  misc_feature (24)..(24): G at position 24 is RNA
aagcagtggtatcaacgcagagtgaatgggtaccaaacgacgagcgtgaca

SEQ ID NO: 2 - DNA, 58 bp, Artificial sequence
  Synthetic
  misc_feature (1)..(58): DNA/RNA Hybrid
  misc_feature (18)..(18): G at position 18 is RNA
  misc_feature (22)..(22): G at position 22 is RNA
  misc_feature (30)..(30): G at position 30 is RNA
gtgactggagttcagacgtgtgctcttccgatctccaatgcttaatcagtgaggcacc

SEQ ID NO: 3 - DNA, 47 bp, Artificial sequence
  Synthetic
  misc_feature (1)..(47): DNA/RNA Hybrid
  misc_feature (17)..(17): G at position 17 is RNA
  misc_feature (20)..(20): G at position 20 is RNA
  misc_feature (24)..(24): G at position 24 is RNA
aagcagtggtatcaacgcagagtgaatgggcccgctgttctgcagcc

SEQ ID NO: 4 - DNA, 53 bp, Artificial sequence
  Synthetic
  misc_feature (1)..(53): DNA/RNA Hybrid
  misc_feature (18)..(18): G at position 18 is RNA
  misc_feature (22)..(22): G at position 22 is RNA
  misc_feature (30)..(30): G at position 30 is RNA
gtgactggagttcagacgtgtgctcttccgatctattcctttgccctcggacg

SEQ ID NO: 5 - DNA, 20 bp, Artificial sequence
  Synthetic
atggcccagtcctctcccaa

SEQ ID NO: 6 - DNA, 22 bp, Artificial sequence
  Synthetic
aggagtaagacccctggaccac

SEQ ID NO: 7 - DNA, 18 bp, Artificial sequence
  Synthetic
agaaccctgaccctgccg

SEQ ID NO: 8 - DNA, 24 bp, Artificial sequence
  Synthetic
ctgaaaaacgtgttcccacccgag

SEQ ID NO: 9 - DNA, 22 bp, Artificial sequence
  Synthetic
acctgaacaaggtgttcccacc

SEQ ID NO: 10 - DNA, 32 bp, Artificial sequence
  Synthetic
  misc_feature (1)..(32): DNA/RNA Hybrid
  misc_feature (16)..(16): G at position 16 is RNA
  misc_feature (18)..(18): G at position 18 is RNA
  misc_feature (20)..(20): G at position 20 is RNA
  misc_feature (28)..(28): G at position 28 is RNA
ttttttttttcagacgtgtgctcttccgatct

SEQ ID NO: 11 - DNA, 33 bp, Artificial sequence
  Synthetic
  misc_feature (1)..(33): DNA/RNA Hybrid
  misc_feature (19)..(19): G at position 19 is RNA
  misc_feature (27)..(27): G at position 27 is RNA
  misc_feature (30)..(30): G at position 30 is RNA
  misc_feature (32)..(32): G at position 32 is RNA
ttttttttttaagcagtggtatcaacgcagagt

SEQ ID NO: 12 - DNA, 23 bp, Artificial sequence
  Synthetic
aagcagtggtatcaacgcagagt

SEQ ID NO: 13 - DNA, 22 bp, Artificial sequence
  Synthetic
cagacgtgtgctcttccgatct

SEQ ID NO: 14 - protein, 130 aa, Aspergillus oryzae
MMYSKLLTLTTLLLPTALALPSLVERACDYTCGSNCYSSSDVSTAQAAGYQLHEDGETVGSNSYPHKYNNYEGFDFSVSSPYYEWPILSSGDVYSGGSPGADRVVFNENNQLAGVITHTGASGNNFVECT

SEQ ID NO: 15 - protein, 150 aa, Bos taurus
MALKSLVLLSLLVLVLLLVRVQPSLGKETAAAKFERQHMDSSTSAASSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

SEQ ID NO: 16 - protein, 198 aa, Escherichia coli
MIEFVYPHTQLVAGVDEVGRGPLVGAVVTAAVILDPARPIAGLNDSKKLSEKRRLALCEEIKEKALSWSLGRAEPHEIDELNILHATMLAMQRAVAGLHIAPEYVLIDGNRCPKLPMPAMAVVKGDSRVPEISAASILAKVTRDAEMAALDIVFPQYGFAQHKGYPTAFHLEKLAEHGATEHHRRSFGPVKRALGLAS

SEQ ID NO: 17 - protein, 155 aa, Escherichia coli
MLKQVEIFTDGSCLGNPGPGGYGAILRYRGREKTFSAGYTRTTNNRMELMAAIVALEALKEHCEVILSTDSQYVRQGITQWIHNWKKRGWKTADKKPVKNVDLWQRLDAALGQHQIKWEWVKGHAGHPENERCDELARAAAMNPTLEDTGYQVEV

SEQ ID NO: 18 - protein, 226 aa, Escherichia coli
MNPIVINRLQRKLGYTFNHQELLQQALTHRSASSKHNERLEFLGDSILSYVIANALYHRFPRVDEGDMSRMRATLVRGNTLAELAREFELGECLRLGPGELKSGGFRRESILADTVEALIGGVFLDSDIQTVEKLILNWYQTRLDEISPGDKQKDPKTRLQEYLQGRHLPLPTYLVVQVRGEAHDQEFTIHCQVSGLSEPVVGTGSSRRKAEQAAAEQALKKLELE

SEQ ID NO: 19 - DNA, 26 bp, Artificial sequence
  Synthetic
acggggagtcaggcaactatggatga

SEQ ID NO: 20 - DNA, 61 bp, Artificial sequence
  Synthetic
  misc_feature (1)..(61): DNA/RNA Hybrid
  misc_feature (27)..(27): G at position 27 is RNA
  misc_feature (30)..(30): G at position 30 is RNA
  misc_feature (34)..(34): G at position 34 is RNA
ttttttttttaagcagtggtatcaacgcagagtgaatgggtaccaaacgacgagcgtgaca

SEQ ID NO: 21 - DNA, 56 bp, Artificial sequence
  Synthetic
  misc_feature (1)..(56): DNA/RNA Hybrid
  misc_feature (16)..(16): G at position 16 is RNA
  misc_feature (20)..(20): G at position 20 is RNA
  misc_feature (28)..(28): G at position 28 is RNA
ttttttttttcagacgtgtgctcttccgatctccaatgcttaatcagtgaggcacc

SEQ ID NO: 22 - DNA, 28 bp, Artificial sequence
  Synthetic
agaagtactcgccgatagtggaaaccga